(19) United States
(12) Patent Application Publication
(10) Pub. No.: US 2024/0407317 A1
(43) Pub. Date: Dec. 12, 2024

(54) PLANTS WITH IMPROVED PROPERTIES

(71) Applicant: BASF SE, Ludwigshafen (DE)

(72) Inventors: Marie-Therese Scheirlinck, Gent (BE); Frank Coen, Gent (BE); Jonny Jacobs, Gent (BE); Ralf-Christian Schmidt, Gent (BE)

(21) Appl. No.: 18/697,521

(22) PCT Filed: Sep. 29, 2022
(86) PCT No.: PCT/EP2022/077216

§ 371 Date: Apr. 1, 2024

(30) Foreign Application Priority Data
Oct. 1, 2021 (EP) 21200484.0

(51) Int. Cl. A01H 6/46; A01H 1/00; A01H 1/06; A01H 5/10

(52) U.S. Cl. CPC A01H 6/4678; A01H 1/06; A01H 1/12; A01H 5/10

(57) ABSTRACT

The present invention relates to wheat plants having an increased yield. More specifically, the invention relates to wheat plants in which expression of ENHANCER OF DA1 (EOD1) is functionally reduced. Provided are wheat plants comprising mutant EOD1 alleles as well as methods and means to produced wheat plants with an increased yield.

### FIELD OF THE INVENTION

This invention relates to wheat plants and parts, with an increased yield. The invention also relates to nucleic acids encoding ENHANCER OF DA1 (EOD1) from wheat and induced variant alleles thereof that affect yield in wheat plants.

### BACKGROUND OF THE INVENTION

Increasing productivity in agriculture is a continuous goal in order to meet the growing demand for food, feed and other plant derived product in view of growing human population and continuous decrease in land space with optimal characteristics which can be allocated to agriculture.

ENHANCER OF DA1 (EOD1, also known as BIG BROTHER, BB) encode RING-finger proteins having E3 ubiquitin-ligase activity which control floral organs and leaf size as well as stem thickness in Arabidopsis (Disch et al, 2006, Current Biology 16, 272-279). Though Li et al (2008, Genes & Dev 22:1331-1336) found that EOD1 can act synergistically with the Ubiquitin receptor DA1 to control seed size, modulating the activity of EOD1 alone does not affect seed size nor seed yield. Some mutations resulting in a reduction or abolishment of EOD1 activity have been described (Li at al 2008, WO2009/047525, WO2015/067943).

There thus remains a need for identifying alleles of EOD1 genes from wheat which will result in wheat plants having an increased yield.

### SUMMARY OF THE INVENTION

In one aspect, the invention provides a wheat plant having a reduced level of EOD1 (ENHANCER OF DA1) gene expression and/or reduced activity of the EOD1 polypeptide compared to a wild type or a control plant. The EOD1 polypeptide may comprise an amino acid sequence selected from the group consisting of (a) the amino acid sequence of any one of SEQ ID NOs: 1, 4, 7; or (b) an amino acid sequence which comprises at least 80% sequence identity to any one of SEQ ID NOs: 1, 4, 7. The EOD1 gene may comprise a nucleic acid sequence selected from the group consisting of (a) the nucleic acid sequence of any one of SEQ ID NOs: 3, 6, 9 and SEQ ID NOs: 2, 5, 8; (b) a nucleic acid sequence having at least 80% sequence identity to any one of SEQ ID NOs: 3, 6, 9 and SEQ ID NOs: 2, 5, 8; (c) a nucleic acid sequence encoding the amino acid sequence of any one of SEQ ID NOs: 1, 4, 7; or (d) a nucleic acid sequence encoding an amino acid sequence which comprises at least 80% sequence identity to any one of SEQ ID NOs: 1, 4, 7.

In a further embodiment, the wheat plant of the invention is characterized by an increase in yield compared to a wild type on control pant. The increased yield may be an increase in grain yield. The increase in grain yield may be an increase in at least one of grain number and/or thousand grain weight.

In another embodiment, the wheat plant of the invention comprises at least one mutation in at least one nucleic acid sequence encoding the EOD1 polypeptide or at least one mutation in the promoter of at least one of the EOD1 gene. The mutation may be an insertion, deletion and/or substitution. The mutation may be a loss of function or partial loss of function mutation and it may be further selected from the group consisting of a) a G to A substitution at a position corresponding to position 3873 of SEQ ID NO: 3; b) a G to A substitution at a position corresponding to position 2992 of SEQ ID NO: 9; and c) a G to A substitution at a position corresponding to position 3873 of SEQ ID NO: 3, and a G to A substitution at a position corresponding to either position 2992 of SEQ ID NO: 9.

The wheat plant of the invention may also comprise a silencing construct that reduces or abolishes the expression of an EOD1 gene and/or reduces or abolishes the activity of the EOD1 polypeptide and/or reduces or abolishes the activity of an EOD1 promoter. The EOD1 promoter may comprise the nucleic acid sequence of any one of SEQ ID NOs: 10 to 12.

The invention furthermore provides a silencing construct capable of suppressing specifically the expression of the endogenous EOD1 gene as described above. Said construct comprises the following operably linked elements (a) a promoter, preferably expressible in plants, (b) a nucleic acid which when transcribed yields an RNA molecule inhibitory to the endogenous EOD1 genes encoding an EOD1 protein; and, optionally (c) a transcription termination and polyadenylation region, preferably a transcription termination and polyadenylation region functional in plants.

The invention further provides a plant cell, plant part or seed of the wheat plant according to the invention. A mutant allele of the above described wheat EOD1 gene is also provided which may comprises the above specified mutations.

In yet another embodiment, a method of increasing yield of a wheat plant compared to a wild type or control wheat plant is provided comprising reducing or abolishing the expression of at least one EOD1 nucleic acid, as described herein, and/or reducing the activity of an EOD1 polypeptide, as described herein, in said plant. A method of producing a wheat plant with increased yield compared to a wild type or control wheat plant is also provided which comprises reducing or abolishing the expression of at least one EOD1 nucleic acid and/or reducing the activity of an EOD1 polypeptide in said plant.

The invention further provides a method for identifying and/or selecting a wheat plant having an increased yield to a wild type or control wheat plant comprising detecting in the plant at least one mutant allele of the invention or at least one mutation in at least one nucleic acid sequence encoding EOD1 or at least one mutation in the promoter of EOD1 resulting in a reduced level of EOD1 gene expression or abolished expression of at least one EOD1 nucleic acid and/or in a reduced activity of an EOD1 polypeptide in said plant compared to a wild type or control wheat plant.

Further provided is the use of a mutant allele of the invention or a loss of function or partial loss of function mutation in at least one nucleic acid sequence encoding EOD1 or at least one mutation in the promoter of EOD1 or of an RNA interference construct that reduces or abolishes the expression of an EOD1 nucleic acid and/or reduces or abolishes the activity of an EOD1 promoter to increase yield of a wheat plant.

Lastly a method of producing food, feed, or an industrial product is provided which comprises (a) obtaining the wheat plant of the invention or a part thereof, and (b) preparing the food, feed or industrial product from the plant or part thereof. The food or feed may be meal, grain, starch, flour or protein. The industrial product may be biofuel, fiber, industrial chemicals, a pharmaceutical or a nutraceutical.

### BRIEF DESCRIPTION OF THE FIGURES

FIG. 1: visualization of the position of the selected and validated wheat EOD1 mutations on the annotated gene sequences: a, representation on the contig A02L6329354 of the structure of the EOD1 gene from the A subgenome, further indicating the position of the A1 mutation; b, representation on the contig B02L8057531 of the structure of the EOD1 gene from the B subgenome; c, representation on the contig D02L9907683 of the structure of the EOD1 gene from the D subgenome, further indicating the position of the D3 mutation; d, conservation of the different EOD1 genes.

FIG. 2: visualization of data showing contrasts (in %) for the different eod1 mutant combinations compared to the corresponding wildtype segregant for: A. YLDHA, B. TKW and C. YLDS. Mutant combinations are as follows: a: EOD1 (A1/-/D1); b: EOD1 (A1/-/-); c: EOD1 (-/-/D3). An asterisk indicates a significant change with p-value<0.05.

### DETAILED DESCRIPTION

The present invention is based on the surprising discovery that loss of function mutations in the wheat EOD1 gene leads to an increased yield.

In one aspect, the invention provides a wheat plant having a reduced level of EOD1 gene expression level of EOD1 (ENHANCER OF DA1) gene expression and/or reduced activity of the EOD1 polypeptide compared to a wild type or a control plant. The EOD1 polypeptide may comprise an amino acid sequence selected from the group consisting of (a) the amino acid sequence of any one of SEQ ID NOs: 1, 4, 7; or (b) an amino acid sequence which comprises at least 80% sequence identity to any one of SEQ ID NOs: 1, 4, 7. The EOD1 gene may comprise a nucleic acid sequence selected from the group consisting of (a) the nucleic acid sequence of any one of SEQ ID NOs: 3, 6, 9 and SEQ ID NOs: 2, 5, 8; (b) a nucleic acid sequence having at least 80% sequence identity to any one of SEQ ID NOs: 3, 6, 9 and SEQ ID NOs: 2, 5, 8; (c) a nucleic acid sequence encoding the amino acid sequence of any one of SEQ ID NOs: 1, 4, 7; or (d) a nucleic acid sequence encoding an amino acid sequence which comprises at least 80% sequence identity to any one of SEQ ID NOs: 1, 4, 7.

“Wheat” or “wheat plant” as used herein can be any variety useful for growing wheat. Examples of wheat are, but are not limited to, Triticum aestivum, Triticum aethiopicum, Triticum Compactum, Triticum dicoccoides, Triticum dicoccon, Triticum durum, Triticum monococcum, Triticum spelta, Triticum turgidum. “Wheat” furthermore encompasses spring and winter wheat varieties, with the winter wheat varieties being defined by a vernalization requirement to flower while the spring wheat varieties do not require such vernalization to flower.

Whenever reference to a “plant” or “plants” according to the invention is made, it is understood that also plant parts (cells, tissues or organs, seed pods, seeds, severed parts such as roots, leaves, flowers, pollen, etc.), progeny of the plants which retain the distinguishing characteristics of the parents, such as seed obtained by selfing or crossing, e.g. hybrid seed (obtained by crossing two inbred parental lines), hybrid plants and plant parts derived there from are encompassed herein, unless otherwise indicated.

In some embodiments, the plant cells of the invention as well as plant cells generated according to the methods of the invention, may be non-propagating cells.

The obtained plants according to the invention can be used in a conventional breeding scheme to produce more plants with the same characteristics or to introduce the same characteristic in other varieties of the same or related plant species, or in hybrid plants. The obtained plants can further be used for creating propagating material. Plants according to the invention can further be used to produce gametes, seeds (including crushed seeds and seed cakes), embryos, either zygotic or somatic, progeny or hybrids of plants obtained by methods of the invention. Seeds obtained from the plants according to the invention are also encompassed by the invention.

“Creating propagating material”, as used herein, relates to any means know in the art to produce further plants, plant parts or seeds and includes inter alia vegetative reproduction methods (e.g. air or ground layering, division, (bud) grafting, micropropagation, stolons or runners, storage organs such as bulbs, corms, tubers and rhizomes, striking or cutting, twin-scaling), sexual reproduction (crossing with another plant) and asexual reproduction (e.g. apomixis, somatic hybridization).

In one aspect, especially in respect of the European Patent Convention, the plant according to the invention is not exclusively obtained by means of an essentially biological process, as for instance defined by Rule 28(2) EPC, or in one aspect the EOD1 mutant allele is not a mutant allele found in the natural population. If such a disclaimer is present in the claim of the European patent, it should be noted that using a plant comprising a mutant allele according to the present invention (e.g. a commercial variety of the applicant) to cross the mutant allele into a different background will still be seen as falling under the claim, even though an exclusively essentially biological process (only crossing and selection) may have been used to transfer the allele into a different background.

The term “gene” means a DNA sequence comprising a region (transcribed region), which is transcribed into an RNA molecule (e.g. into a pre-mRNA, comprising intron sequences, which is then spliced into a mature mRNA, or directly into a mRNA without intron sequences) in a cell, operable linked to regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites.

The term “EOD1 gene” as used herein refers to a nucleotide sequence encoding the ENHANCER OF DA1 protein, which is a protein having E3 ubiquitin-ligase activity and is therefore likely involved in marking proteins for degradation (Disch et al 2006). It comprises a RING-finger domain of the H2 type, also known as a LIM domain (Prosite: PS00478), at its C-terminus at position equivalent to positions 206 to 247 of any one of SEQ ID NOs: 1, 4 and 7 (WO 2015/067943).

The phrases “DNA”, “DNA sequence,” “nucleic acid sequence,” “nucleic acid molecule” “nucleotide sequence” and “nucleic acid” refer to a physical structure comprising an orderly arrangement of nucleotides. The DNA sequence or nucleotide sequence may be contained within a larger nucleotide molecule, vector, or the like. In addition, the orderly arrangement of nucleic acids in these sequences may be depicted in the form of a sequence listing, figure, table, electronic medium, or the like.

The EOD1 gene described herein and used in the methods of the present invention is in one embodiment a EOD1 gene having at least about 70%, at least about 72%, at least about 74%, at least about 76%, at least about 78%, at least about 80%, at least about 82%, at least about 84%, at least about 86%, at least about 88%, at least about 90%, at least about 91%, at least about 92%, at least about 93%, at least about 94%, at least about 95%, at least about 96%, at least about 97%, at least about 98%, at least about 99%, and at least about 100% identity with any one of SEQ ID NOs: 2, 5, 8 and SEQ ID NOs: 3, 6, 9.

Sequence identity usually is provided as “% sequence identity” or “% identity”. To determine the percent-identity between two nucleic acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters for nucleic acid alignments (gapopen=10.0, gapextend=0.5 and matrix=EDNAFULL).

The following example is meant to illustrate alignment for two nucleotide sequences:

Seq A:
AAGATACTG length: 9 bases
Seq B:
GATCTGA length: 7 bases

Hence, the shorter sequence is sequence B.

Producing a pairwise global alignment which is showing both sequences over their complete lengths results in

| | |
|---|---|
| Seq A: | AAGATACTG- |
| | \|\|\| \|\|\| |
| Seq B: | --GAT-CTGA |

The “I” symbol in the alignment indicates identical residues (which means bases for DNA or amino acids for proteins). The number of identical residues is 6. The “-” symbol in the alignment indicates gaps. The number of gaps introduced by alignment within the Seq B is 1. The number of gaps introduced by alignment at borders of Seq B is 2, and at borders of Seq A is 1. The alignment length showing the aligned sequences over their complete length is 10.

Producing a pairwise alignment which is showing the shorter sequence over its complete length according to the invention consequently results in:

| | |
|---|---|
| Seq A: | GATACTG- |
| | \|\|\| \|\|\| |
| Seq B: | GAT-CTGA |

Producing a pairwise alignment which is showing sequence A over its complete length according to the invention consequently results in:

| | |
|---|---|
| Seq A: | AAGATACTG |
| | \|\|\| \|\|\| |
| Seq B: | --GAT-CTG |

Producing a pairwise alignment which is showing sequence B over its complete length according to the invention consequently results in:

| | |
|---|---|
| Seq A: | GATACTG- |
| | \|\|\| \|\|\| |
| Seq B: | GAT-CTGA |

The alignment length showing the shorter sequence over its complete length is 8 (one gap is present which is factored in the alignment length of the shorter sequence). Accordingly, the alignment length showing Seq A over its complete length would be 9 (meaning Seq A is the sequence of the invention). Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the respective sequence of this invention over its complete length)*100. Thus, sequence identity in relation to comparison of two nucleic acid sequences according to this embodiment is calculated by dividing the number of identical residues by the length of the alignment region which is showing the respective sequence of this invention over its complete length. This value is multiplied with 100 to give “%-identity”. According to the example provided above, %-identity is: for Seq A being the sequence of the invention (6/9)*100=66.7%; for Seq B being the sequence of the invention (6/8)*100=75%.

For nucleic acid sequences encoding for a protein or a peptide, the pairwise alignment shall be made over the complete length of the coding region of the sequence of this invention. Introns present in the other sequence may be removed for the pairwise alignment to allow comparison with the sequence of this invention. Percent identity is then calculated by: %-identity=(identical residues/length of the alignment region which is showing the coding region of the sequence of this invention over its complete length)*100.

“Expression of a gene” or “gene expression” refers to the process wherein a DNA region, which is operably linked to appropriate regulatory regions, particularly a promoter, is transcribed into an RNA molecule. The RNA molecule is then processed further (by post-transcriptional processes) within the cell, e.g. by RNA splicing and translation initiation and translation into an amino acid chain (protein), and translation termination by translation stop codons. The term “functionally expressed” is used herein to indicate that a functional protein is produced; the term “not functionally expressed” to indicate that a protein with significantly reduced level of EOD1 gene expression or no functionality (biological activity) is produced or that no protein is produced (see further below).

“A reduced level of EOD1 gene expression” refers to a reduction in the amount of RNA molecule transcribed from the EOD1 gene which may be translated into a functional EOD1 protein by at least 30%, 40%, 50%, 60%, 70%, 80%, 90%, or 100% compared to the amount of RNA molecule transcribed from the EOD1 gene which may be translated into a functional EOD1 protein in a wild type or control plant.

The term “protein” interchangeably used with the term “polypeptide” as used herein describes a group of molecules consisting of more than 30 amino acids, whereas the term “peptide” describes molecules consisting of up to 30 amino acids. Proteins and peptides may further form dimers, trimers and higher oligomers, i.e. consisting of more than one (poly) peptide molecule. Protein or peptide molecules forming such dimers, trimers etc. may be identical or non-identical. The corresponding higher order structures are, consequently, termed homo- or heterodimers, homo- or heterotrimers etc. The terms “protein” and “peptide” also refer to naturally modified proteins or peptides wherein the modification is obtained e.g. by glycosylation, acetylation, phosphorylation and the like. Such modifications are well known in the art.

A “reduced activity of an EOD1 polypeptide” refers to a reduction in the amount of a functional EOD1 protein produced by at least 30%, 40%, 50%, 60%, 70%, 80%, 90%, or 100% compared to the amount of functional EOD1 protein produced in a wild type or control plant. This definition encompasses the production of a “non-functional” EOD1 protein (e.g. truncated EOD1protein) resulting from reduced level of EOD1 gene expression biological activity in vivo, the reduction in the absolute amount of the functional EOD1 protein (e.g. no functional EOD1 protein being made due to the mutation in the EOD1 gene), the production of an EOD1 protein with significantly reduced level of EOD1 biological activity compared to the activity of a functional wild type EOD1 protein (such as an EOD1 protein in which one or more amino acid residues that are crucial for the biological activity of the encoded EOD1 protein are substituted for another amino acid residue).

“Wild type” (also written “wildtype” or “wild-type”) or “control”, as used herein, refers to a typical form of a plant or a gene as it most commonly occurs in nature. A “wild type plant” or “control plant” refers to a plant with the most common genotype at the EOD1 loci in the natural population.

The EOD1 protein described herein and used in the methods of the present invention is in one embodiment a EOD1 protein having at least about 70%, at least about 72%, at least about 74%, at least about 76%, at least about 78%, at least about 80%, at least about 82%, at least about 84%, at least about 86%, at least about 88%, at least about 90%, at least about 91%, at least about 92%, at least about 93%, at least about 94%, at least about 95%, at least about 96%, at least about 97%, at least about 98%, at least about 99%, and at least about 100% identity with any one of SEQ ID NOs: 1, 4, 7.

To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined. The same calculations apply as those of the example above illustrating two nucleotide sequences.

After aligning two sequences, in a second step, an identity value is determined from the alignment produced. For purposes of this description, percent identity is calculated by %-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”. According to the example provided above, %-identity is: for Seq A being the sequence of the invention (6/9)*100=66.7%; for Seq B being the sequence of the invention (6/8)*100=75%.

In a further embodiment, the wheat plant of the invention is characterized by an increase in yield compared to a wild type on control pant. The increased yield may be an increase in grain yield. The increase in grain yield may be an increase in at least one of grain number and/or thousand grain weight.

“Yield” as used herein can comprise yield of the plant or plant part which is harvested, such as grain, grain protein content, grain weight (measured as thousand grain weight, i.e. the weight of one thousand grains), grain number. Increased yield can be increased yield per plant, and increased yield per surface unit of cultivated land, such as yield per hectare. Yield can be increased by modulating, for example, by increasing seed size or indirectly by increasing the tolerance to biotic and abiotic stress conditions and decreasing seed abortion.

When the yield is the grain yield, the yield increase achieved with the method described herein compared to wild type or control wheat plant may be of at least about 2.5%, at least about 3%, at least about 3.5%, at least about 4% at least about 4.5%, at least about 5%, at least about 6%, at least about 7%, at least about 8%, at least about 9%, at least about 10%, at least about 11%, at least about 12%, at least 13%, at least 14%, at least 15% or at least 20%. When the yield is the grain weight, the yield increase achieved with the method described herein compared to wild type or control wheat plant may be of at least about 2.5%, at least about 3%, at least about 3.5%, at least about 4% at least about 4.5%, at least about 5%, at least about 6%, at least about 7% or at least about 8%, at least about 9%, at least about 10%, at least about 11%, at least about 12%, at least 13%, at least 14%, at least 15% or at least 20%. When the yield is the grain number, the yield increase achieved with the method described herein compared to wild type or control wheat plant may be of at least about 2.5%, at least about 3%, at least about 3.5%, at least about 4% at least about 4.5%, at least about 5%, at least about 6%, at least about 7% or at least about 8%, at least about 9%, at least about 10%, at least about 11%, at least about 12%, at least 13%, at least 14%, at least 15% or at least 20%.

In another embodiment, the wheat plant of the invention comprises at least one mutation in at least one nucleic acid sequence encoding the EOD1 polypeptide or at least one mutation in the promoter of at least one of the EOD1 gene. The mutation may be an insertion, deletion and/or substitution. The mutation may be a loss of function or partial loss of function mutation and it may be further selected from the group consisting of a) a G to A substitution at a position corresponding to position 3873 of SEQ ID NO: 3; b) a G to A substitution at a position corresponding to position 2992 of SEQ ID NO: 9; and c) a G to A substitution at a position corresponding to position 3873 of SEQ ID NO: 3, and a G to A substitution at a position corresponding to either position 2992 of SEQ ID NO: 9.

The mutation in a nucleic acid sequence encoding the EOD1 polypeptide or in the promoter of an EOD1 gene can be created by mutagenesis or by gene editing.

“Mutagenesis”, as used herein, refers to the process in which plant cells (e.g., a plurality of cereal seeds or other parts, such as pollen, etc.) are subjected to a technique which induces mutations in the DNA of the cells, such as contact with a mutagenic agent, such as a chemical substance (such as ethylmethylsulfonate (EMS), ethylnitrosourea (ENU), etc.) or ionizing radiation (neutrons (such as in fast neutron mutagenesis, etc.), alpha rays, gamma rays (such as that supplied by a Cobalt 60 source), X-rays, UV-radiation, etc.), T-DNA insertion mutagenesis (Azpiroz-Leehan et al. (1997) Trends Genet 13:152-156), transposon mutagenesis (Mckenzie et al. (2002) Theor Appl Genet 105:23-33), or tissue culture mutagenesis (induction of somaclonal variations), or a combination of two or more of these. While mutations created by irradiation are often large deletions or other gross lesions such as translocations or complex rearrangements, mutations created by chemical mutagens are often more discrete lesions such as point mutations. For example, EMS alkylates guanine bases, which results in base mispairing: an alkylated guanine will pair with a thymine base, resulting primarily in G/C to A/T transitions. Following mutagenesis, wheat plant plants are regenerated from the treated cells using known techniques. For instance, the resulting wheat seeds (or wheat grain) may be planted in accordance with conventional growing procedures and following self-pollination seed is formed on the plants. Additional seed (or grain) that is formed as a result of such self-pollination in the present or a subsequent generation may be harvested and screened for the presence of the mutation in a nucleic acid sequence encoding the EOD1 polypeptide or in the promoter of a EOD1 gene or of mutant eod1 alleles. Several techniques are known to screen for specific mutations in a gene or mutant alleles, e.g., Deleteagene™ (Delete-a-gene; Li et al., 2001, Plant J 27:235-242) uses polymerase chain reaction (PCR) assays to screen for deletion mutants generated by fast neutron mutagenesis, TILLING (targeted induced local lesions in genomes; McCallum et al., 2000, Nat Biotechnol 18:455-457) identifies EMS-induced point mutations, etc.

“Gene editing”, as used herein, refers to the targeted modification of genomic DNA using sequence-specific enzymes (such as endonuclease, nickases, base conversion enzymes) and/or donor nucleic acids (e.G. dsDNA, oligo's) to introduce desired changes in the DNA. Sequence-specific nucleases that can be programmed to recognize specific DNA sequences include meganucleases (MGNs), zinc-finger nucleases (ZFNs), TAL-effector nucleases (TALENs) and RNA-guided or DNA-guided nucleases such as Cas9, Cpf1, CasX, CasY, C2c1, C2c3, certain Argonaut-based systems (see e.G. Osakabe and Osakabe, Plant Cell Physiol. 2015 March; 56 (3): 389-400; Ma et al., Mol Plant. 2016 Jul. 6; 9 (7): 961-74; Bortesie et al., Plant Biotech J, 2016, 14; Murovec et al., Plant Biotechnol J. 2017 Apr. 1; Nakade et al., Bioengineered 8-3, 2017; Burstein et al., Nature 542, 37-241; Komor et al., Nature 533, 420-424, 2016; all incorporated herein by reference). Donor nucleic acids can be used as a template for repair of the DNA break induced by a sequence specific nuclease but can also be used as such for gene targeting (without DNA break induction) to introduce a desired change into the genomic DNA. Sequence-specific nucleases may also be used without donor nucleic acid, thereby allowing insertion or deletion mutations via non homologous end joining repair mechanism.

Mutant nucleic acid molecules or mutant alleles may comprise one or more mutations or modifications, such as:
1. a “missense mutation”, which is a change in the nucleic acid sequence that results in the substitution of an amino acid for another amino acid;
2. a “nonsense mutation” or “STOP codon mutation”, which is a change in the nucleic acid sequence that results in the introduction of a premature STOP codon and thus the termination of translation (resulting in a truncated protein); plant genes contain the translation stop codons “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation;
3. an “insertion mutation” of one or more amino acids, due to one or more codons having been added in the coding sequence of the nucleic acid;
4. a “deletion mutation” of one or more amino acids, due to one or more codons having been deleted in the coding sequence of the nucleic acid;
5. a “frameshift mutation”, resulting in the nucleic acid sequence being translated in a different frame downstream of the mutation. A frameshift mutation can have various causes, such as the insertion, deletion or duplication of one or more nucleotides;
6. a mutated splice site, resulting in altered splicing, which results in an altered mRNA processing and, consequently, in an altered encoded protein which contains either deletions, substitutions or insertions of various lengths, possibly combined with premature translation termination.

Mutations in a nucleic acid sequence encoding the EOD1 polypeptide or in the promoter of an EOD1 gene are provided herein which may be loss of function mutations or partial loss of function mutations.

A “loss of function mutation”, as used herein, refers to a mutation in a gene, which results in said gene encoding a protein having no biological activity as compared to the corresponding wild-type functional protein or which encodes no protein at all. Such a “loss of function” mutation is, for example, one or more non-sense, missense, insertion, deletion, frameshift or mutated splice site mutations. In particular, such a loss of function mutation in an EOD1 gene may be a mutation that preferably results in the production of an EOD1 protein lacking at least one functional domain or motif, such as the RING-finger domain of the H2 type, also known as a LIM domain (Prosite: PS00478), such that the biological activity of the EOD1 protein is completely abolished, or whereby the modification(s) preferably result in no production of an EOD1 protein.

A “partial loss of function mutation”, as used herein, refers to a mutation in a gene, which results in said gene encoding a protein having a significantly reduced level of EOD1 biological activity as compared to the corresponding wild-type functional protein. Such a “partial loss of function mutation” is, for example, one or more mutations in the nucleic acid sequence of the gene, for example, one or more missense mutations. In particular, such a partial loss of function mutation is a mutation that preferably results in the production of a protein wherein at least one conserved and/or functional amino acid is substituted for another amino acid, such that the biological activity is significantly reduced level of EOD1 gene expression but not completely abolished, or results in the production of an EOD1 protein lacking at least part of a functional domain or motif, such as part of the RING-finger domain of the H2 type, also known as a LIM domain (Prosite: PS00478), such that the biological activity of the EOD1 protein is reduced.

A missense mutation in a EOD1 gene, as used herein, is any mutation (deletion, insertion or substitution) in a EOD1 gene whereby one or more codons are changed into the coding DNA and the corresponding mRNA sequence of the corresponding wild type EOD1 allele, resulting in the substitution of one or more amino acids in the wild type EOD1 protein for one or more other amino acids in the mutant EOD1 protein. An EOD1 mutant allele comprising a missense mutation is an EOD1 allele wherein one amino acid is substituted.

A nonsense mutation in an EOD1gene, as used herein, is a mutation in an EOD1 allele whereby one or more translation stop codons are introduced into the coding DNA and the corresponding mRNA sequence of the corresponding wild type EOD1 allele. Translation stop codons are TGA (UGA in the mRNA), TAA (UAA) and TAG (UAG). Thus, any mutation (deletion, insertion or substitution) that leads to the generation of an in-frame stop codon in the coding sequence will result in termination of translation and truncation of the amino acid chain. The truncated protein lacks the amino acids encoded by the coding DNA downstream of the mutation (i.e. the C-terminal part of the EOD1 protein) and maintains the amino acids encoded by the coding DNA upstream of the mutation (i.e. the N-terminal part of the EOD1 protein). The more truncated the mutant EOD1 protein is in comparison to the wild type EOD1 protein, the more the truncation may result in a significantly reduced activity of the EOD1 protein. It is believed that, in order for the mutant EOD1 protein to lose some biological activity, it should at least no longer comprise the complete RING-finger domain of the H2 type, also known as a LIM domain (Prosite: PS00478) at the amino acid positions equivalent to position [ . . . ] of SEQ ID NOs: 2, 5, 8.

A frameshift mutation in an EOD1 gene, as used herein, is a mutation (deletion, insertion, duplication, and the like) in an EOD1 allele that results in the nucleic acid sequence being translated in a different frame downstream of the mutation.

A splice site mutation in a EOD1 gene, as used herein, is a mutation (deletion, insertion, substitution, duplication, and the like) in an EOD1 allele whereby a splice donor site or a splice acceptor site is mutated, resulting in altered processing of the mRNA and, consequently, an altered encoded protein, which can have insertions, deletions, substitutions of various lengths, or which can be truncated.

A deletion mutation in a EOD1 gene, as used herein, is a mutation in a EOD1 gene that results in the production of a EOD1 protein which lacks the amino acids encoded by the deleted coding DNA and maintains the amino acids encoded by the coding DNA upstream of the deletion (i.e. the N-terminal part of the EOD1 protein) and encoding by the coding DNA downstream of the deletion (i.e. the C-terminal part of the EOD1 protein).

TABLE 1
Examples of substitution mutation resulting in the generation of an
in-frame stop codon or splice site mutation.

| position on any one of | codon before | codon after | |
|---|---|---|---|
| SEQ ID NOs: 2, 5, 8 | substitution | substitution | consequence |
| 708 | TGG | TGA | nonsense |
| 707 | TGG | TAG | nonsense |
| 688 | CGA | TGA | nonsense |
| 680 | CAG | TAG | nonsense |
| 673 | CAG | TAG | nonsense |
| 499 | CAA | TAA | nonsense |
| 196 | CAA | TAA | nonsense |
| 442 | CAG | TAG | nonsense & |
| | | | splice site |
| 337 | CAA | TAA | nonsense |
| 322 | CAG | TAG | nonsense |
| 286 | CAG | TAG | nonsense |
| 253 | CAG | TAG | nonsense |
| 238 | CAG | TAG | nonsense |
| 172 | CAA | TAA | nonsense |
| 163 | CAA | TAA | nonsense |
| 148 | CAG | TAG | nonsense & |
| | | | splice site |
| 142 | CAA | TAA | nonsense |
| 133 | CAA | TAA | nonsense |

As used herein, “promoter” means a region of DNA sequence that is essential for the initiation of transcription of DNA, resulting in the generation of an RNA molecule that is complementary to the transcribed DNA; this region may also be referred to as a “5′ regulatory region.” Promoters are usually located upstream of the coding sequence to be transcribed and have regions that act as binding sites for RNA polymerase II and other proteins such as transcription factors (trans-acting protein factors that regulate transcription) to initiate transcription of an operably linked gene. Promoters may themselves contain sub-elements (i.e. promoter motifs) such as cis-elements or enhancer domains that regulate the transcription of operably linked genes. The promoters of this invention may be altered to remove “enhancer DNA” to assist in reduced level of EOD1 gene expression gene expression. As is known in the art, certain DNA elements (“enhancer DNA”) can be used to enhance the transcription of DNA. These enhancers often are found 5′ to the start of transcription in a promoter that functions in eukaryotic cells but can often be upstream (5′) or downstream (3′) to the coding sequence. In some instances, these 5′ enhancer DNA elements are introns. The promoters may also be altered to remove DNA known to be essential to a promoter activity like for example the TATA box, the activity of the promoter is then abolished. Thus, as contemplated herein, a promoter or promoter region includes variations of promoters derived by inserting or deleting regulatory regions, subjecting the promoter to random or site-directed mutagenesis, etc. The activity or strength of a promoter may be measured in terms of the amounts of RNA it produces, or the amount of protein accumulation in a cell or tissue, relative to a promoter whose transcriptional activity has been previously assessed. A promoter as used herein may thus include sequences downstream of the transcription start, such as sequences coding the 5′ untranslated region (5′ UTR) of the RNA, introns located downstream of the transcription start, or even sequences encoding the protein.

Suitable for the invention are wheat plants comprising at least one mutation in at least one, at least two, at least three, at least four, at least five or even in all six nucleic acid sequences encoding the EOD1 polypeptide. Such at least one mutation in at least one nucleic acid sequence encoding the EOD1 polypeptide is equivalent to at least one EOD1 mutant allele and may be at least one EOD1 mutant allele from the subgenome A, at least one EOD1 mutant allele from the subgenome B or at least one EOD1 mutant allele from the subgenome D. Such at least one mutation in at least two nucleic acid sequence encoding the EOD1 polypeptide is equivalent to at least two EOD1 mutant alleles and may be two EOD1 mutant alleles from the subgenome B, two EOD1 mutant alleles from the subgenome D, two EOD1 mutant alleles from the subgenome A, at least one EOD1 mutant allele from the subgenome B and at least one EOD1 mutant allele from the subgenome D, at least one EOD1 mutant allele from the subgenome B and at least one EOD1 mutant allele from the subgenome A or at least one EOD1 mutant allele from the subgenome D and at least one EOD1 mutant allele from the subgenome A. Such at least one mutation in at least three nucleic acid sequences encoding the EOD1 polypeptide is equivalent to at least three EOD1 mutant alleles and may be two EOD1 mutant alleles from the subgenome B and at least one EOD1 mutant allele from the subgenome A, two EOD1 mutant alleles from the subgenome B and at least one EOD1 mutant allele from the subgenome D, two EOD1 mutant alleles from the subgenome D and at least one EOD1 mutant allele from the subgenome B, two EOD1 mutant alleles from the subgenome D and at least one EOD1 mutant allele from the subgenome A, two EOD1 mutant alleles from the subgenome A and at least one EOD1 mutant allele from the subgenome B, two EOD1 mutant alleles from the subgenome A and at least one EOD1 mutant allele from the subgenome D or at least one EOD1 mutant allele from the subgenome B, at least one EOD1 mutant allele from the subgenome A and at least one EOD1 mutant allele from the subgenome D.

Such at least one mutation in at least four nucleic acid sequences encoding the EOD1 polypeptide is equivalent to at least four EOD1 mutant alleles and may be two EOD1 mutant alleles from the subgenome B and two EOD1 mutant alleles from the subgenome A, two EOD1 mutant alleles from the subgenome B and two EOD1 mutant alleles from the subgenome D, or two EOD1 mutant alleles from the subgenome D and two EOD1 mutant alleles from the subgenome A. They may also be two EOD1 mutant alleles from the subgenome B, at least one EOD1 mutant allele from the subgenome A and at least one EOD1 mutant allele from the subgenome D, or two EOD1 mutant alleles from the subgenome D, at least one EOD1 mutant allele from the subgenome A and at least one EOD1 mutant allele from the subgenome B, or two EOD1 mutant alleles from the subgenome A, at least one EOD1 mutant allele from the subgenome B and at least one EOD1 mutant allele from the subgenome D. Such at least one mutation in at least five nucleic acid sequences encoding the EOD1 polypeptide is equivalent to at least five EOD1 mutant alleles and may be two EOD1 mutant alleles from the subgenome B, two EOD1 mutant alleles from the subgenome A and at least one EOD1 mutant allele from the subgenome D, or two EOD1 mutant alleles from the subgenome B, two EOD1 mutant alleles from the subgenome D and at least one EOD1 mutant allele from the subgenome A, or two EOD1 mutant alleles from the subgenome D, two EOD1 mutant alleles from the subgenome A and at least one EOD1 mutant allele from the subgenome B. Such at least one mutation in all six nucleic acid sequences encoding the EOD1 polypeptide is equivalent to six EOD1 mutant alleles and may be two EOD1 mutant alleles from the subgenome D, two EOD1 mutant alleles from the subgenome A and two EOD1 mutant alleles from the subgenome B.

Also suitable for the invention are wheat plants comprising at least one mutation in the promoter of at least one, at least two or in all three of the EOD1 genes. Such at least one mutation in the promoter of at least one EOD1 gene may be equivalent to at least one EOD1 mutant allele in which case it may be at least one EOD1 mutant allele from the subgenome A, at least one EOD1 mutant allele from the subgenome B or at least one EOD1 mutant allele from the subgenome D, or it may be equivalent to at least two EOD1 mutant alleles in which case it may be two EOD1 mutant alleles from the subgenome B, two EOD1 mutant alleles from the subgenome D, two EOD1 mutant alleles from the subgenome A. Such at least one mutation in the promoter of at least two EOD1 gene may be equivalent to at least two EOD1 mutant alleles in which case it may be at least one EOD1 mutant allele from the subgenome B and at least one EOD1 mutant allele from the subgenome D, at least one EOD1 mutant allele from the subgenome B and at least one EOD1 mutant allele from the subgenome A or at least one EOD1 mutant allele from the subgenome D and at least one EOD1 mutant allele from the subgenome A, it may be equivalent to at least three EOD1 mutant alleles in which case it may be two EOD1 mutant alleles from the subgenome B and at least one EOD1 mutant allele from the subgenome A, two EOD1 mutant alleles from the subgenome B and at least one EOD1 mutant allele from the subgenome D, two EOD1 mutant alleles from the subgenome D and at least one EOD1 mutant allele from the subgenome B, two EOD1 mutant alleles from the subgenome D and at least one EOD1 mutant allele from the subgenome A, two EOD1 mutant alleles from the subgenome A and at least one EOD1 mutant allele from the subgenome B, two EOD1 mutant alleles from the subgenome A and at least one EOD1 mutant allele from the subgenome D, or it may be equivalent to four EOD1 mutant alleles in which case it may be two EOD1 mutant alleles from the subgenome B and two EOD1 mutant alleles from the subgenome A, two EOD1 mutant alleles from the subgenome B and two EOD1 mutant alleles from the subgenome D, or two EOD1 mutant alleles from the subgenome D and two EOD1 mutant alleles from the subgenome A. Such at least one mutation in the promoter of all three EOD1 genes may be equivalent to at least three EOD1 mutant alleles in which case it may be at least one EOD1 mutant allele from the subgenome B, at least one EOD1 mutant allele from the subgenome A and at least one EOD1 mutant allele from the subgenome D, it may be equivalent to at least four EOD1 mutant alleles in which case it may be two EOD1 mutant alleles from the subgenome B, at least one EOD1 mutant allele from the subgenome A and at least one EOD1 mutant allele from the subgenome D, or two EOD1 mutant alleles from the subgenome D, at least one EOD1 mutant allele from the subgenome A and at least one EOD1 mutant allele from the subgenome B, or two EOD1 mutant alleles from the subgenome A, at least one EOD1 mutant allele from the subgenome B and at least one EOD1 mutant allele from the subgenome D, it may also be equivalent to at least five EOD1 mutant alleles in which case it may be two EOD1 mutant alleles from the subgenome B, two EOD1 mutant alleles from the subgenome A and at least one EOD1 mutant allele from the subgenome D, or two EOD1 mutant alleles from the subgenome B, two EOD1 mutant alleles from the subgenome D and at least one EOD1 mutant allele from the subgenome A, or two EOD1 mutant alleles from the subgenome D, two EOD1 mutant alleles from the subgenome A and at least one EOD1 mutant allele from the subgenome B, or it may be equivalent to six EOD1 mutant alleles and may then be two EOD1 mutant alleles from the subgenome D, two EOD1 mutant alleles from the subgenome A and two EOD1 mutant alleles from the subgenome B.

The wheat plant of the invention may also comprise a silencing construct that reduces or abolishes the expression of an EOD1 gene and/or reduces or abolishes the activity of an EOD1 promoter. The EOD1 promoter may comprise the nucleic acid sequence of any one of SEQ ID NOs: 10 to 12.

The term “construct” refers to any artificial gene that contains: a) DNA sequences, including regulatory and coding sequences that are not found together in nature, or b) sequences encoding parts of proteins not naturally adjoined, or c) parts of promoters that are not naturally adjoined. Accordingly, a construct may comprise regulatory sequences and coding sequences that are derived from different sources, i.e. heterologous sequences, or comprise regulatory sequences, and coding sequences derived from the same source, but arranged in a manner different from that found in nature.

The term “heterologous” refers to the relationship between two or more nucleic acid or protein sequences that are derived from different sources. For example, a promoter is heterologous with respect to an operably linked DNA region, such as a coding sequence if such a combination is not normally found in nature. In addition, a particular sequence may be “heterologous” with respect to a cell or organism into which it is inserted (i.e. does not naturally occur in that particular cell or organism). For example, the construct disclosed herein is a heterologous nucleic acid.

The invention furthermore provides a silencing construct capable of suppressing specifically the expression of the endogenous EOD1 genes as described above. Said construct comprises the following operably linked elements (a) a promoter, preferably expressible in plants, (b) a nucleic acid which when transcribed yields an RNA molecule inhibitory to the endogenous EOD1 genes encoding an EOD1 protein; and, optionally (c) a transcription termination and polyadenylation region, preferably a transcription termination and polyadenylation region functional in plants.

Such inhibitory RNA molecule can reduce the expression of a gene for example through the mechanism of RNA-mediated gene silencing. It can be a silencing RNA downregulating expression of a target gene. As used herein, “silencing RNA” or “silencing RNA molecule” refers to any RNA molecule, which upon introduction into a plant cell, reduces the expression of a target gene. Such silencing RNA may e.g. be so-called “antisense RNA”, whereby the RNA molecule comprises a sequence of at least 20 consecutive nucleotides having 95% sequence identity to the complement of the sequence of the target nucleic acid, preferably the coding sequence of the target gene. However, antisense RNA may also be directed to regulatory sequences of target genes, including the promoter sequences and transcription termination and polyadenylation signals. Silencing RNA further includes so-called “sense RNA” whereby the RNA molecule comprises a sequence of at least 20 consecutive nucleotides having 95% sequence identity to the sequence of the target nucleic acid. Other silencing RNA may be “unpolyadenylated RNA” comprising at least 20 consecutive nucleotides having 95% sequence identity to the complement of the sequence of the target nucleic acid, such as described in WO01/12824 or U.S. Pat. No. 6,423,885 (both documents herein incorporated by reference). Yet another type of silencing RNA is an RNA molecule as described in WO03/076619 (herein incorporated by reference) comprising at least 20 consecutive nucleotides having 95% sequence identity to the sequence of the target nucleic acid or the complement thereof, and further comprising a largely-double stranded region as described in WO03/076619 (including largely double stranded regions comprising a nuclear localization signal from a viroid of the Potato spindle tuber viroid-type or comprising CUG trinucleotide repeats). Silencing RNA may also be double stranded RNA comprising a sense and antisense strand as herein defined, wherein the sense and antisense strand are capable of base-pairing with each other to form a double stranded RNA region (preferably the said at least 20 consecutive nucleotides of the sense and antisense RNA are complementary to each other). The sense and antisense region may also be present within one RNA molecule such that a hairpin RNA (hpRNA) can be formed when the sense and antisense region form a double stranded RNA region. hpRNA is well-known within the art (see e.G WO99/53050, herein incorporated by reference). The hpRNA may be classified as long hpRNA, having long, sense and antisense regions which can be largely complementary, but need not be entirely complementary (typically larger than about 200 bp, ranging between 200-1000 bp). hpRNA can also be rather small ranging in size from about 30 to about 42 bp, but not much longer than 94 bp (see WO04/073390, herein incorporated by reference). Silencing RNA may also be artificial micro-RNA molecules as described e.G. in WO2005/052170, WO2005/047505 or US 2005/0144667, or ta-siRNAs as described in WO2006/074400 (all documents incorporated herein by reference). Said RNA capable of modulating the expression of a gene can also be an RNA ribozyme.

The phrase “operably linked” refers to the functional spatial arrangement of two or more nucleic acid regions or nucleic acid sequences. For example, a promoter region may be positioned relative to a nucleic acid sequence such that transcription of a nucleic acid sequence is directed by the promoter region. Thus, a promoter region is “operably linked” to the nucleic acid sequence. “Functionally linked” is an equivalent term.

A “transcription termination and polyadenylation region” as used herein is a sequence that controls the cleavage of the nascent RNA, whereafter a poly (A) tail is added at the resulting RNA 3′ end, functional in plant cells. Transcription termination and polyadenylation signals functional in plant cells include, but are not limited to, 3′nos, 3′35S, 3′his and 3′g7.

As used herein, the term “plant-expressible promoter” means a promoter that is capable of controlling (initiating) transcription in a plant cell. This includes any promoter of plant origin, but also any promoter of non-plant origin which is capable of directing transcription in a plant cell, i.e., certain promoters of viral or bacterial origin such as the CaMV35S (Harpster et al. (1988) Mol Gen Genet. 212 (1): 182-90, the subterranean clover virus promoter No 4 or No 7 (WO9606932), or T-DNA gene promoters but also tissue-specific or organ-specific promoters including but not limited to seed-specific promoters (e.G., WO89/03887), organ-primordia specific promoters (An et al. (1996) Plant Cell 8 (1): 15-30), stem-specific promoters (Keller et al., (1988) EMBO J. 7 (12): 3625-3633), leaf specific promoters (Hudspeth et al. (1989) Plant Mol Biol. 12:579-589), mesophyl-specific promoters (such as the light-inducible Rubisco promoters), root-specific promoters (Keller et al. (1989) Genes Dev. 3:1639-1646), tuber-specific promoters (Keil et al. (1989) EMBO J. 8 (5): 1323-1330), vascular tissue specific promoters (Peleman et al. (1989) Gene 84:359-369), stamen-selective promoters (WO 89/10396, WO 92/13956), dehiscence zone specific promoters (WO 97/13865) and the like.

Suitable plant-expressible promoters for the invention are constitutive plant-expressible promoters. Constitutive plant-expressible promoters are well known in the art and include the CaMV35S promoter (Harpster et al. (1988) Mol Gen Genet. 212 (1): 182-90), Actin promoters, such as, for example, the promoter from the Rice Actin gene (McElroy et al., 1990, Plant Cell 2:163), the promoter of the Cassava Vein Mosaic Virus (Verdaguer et al., 1996 Plant Mol. Biol. 31:1129), the GOS promoter (de Pater et al., 1992, Plant J. 2:837), the Histone H3 promoter (Chaubet et al., 1986, Plant Mol Biol 6:253), the Agrobacterium tumefaciens Nopaline Synthase (Nos) promoter (Depicker et al., 1982, J. Mol. Appl. Genet. 1:561), or Ubiquitin promoters, such as, for example, the promoter of the maize Ubiquitin-1 gene (Christensen et al., 1992, Plant Mol. Biol. 18:675).

A further promoter suitable for the invention is the endogenous promoter driving expression of the gene encoding a EOD1 protein.

The term “endogenous” relates to what originate from within the plant or cell. An endogenous gene, promoter or allele is thus respectively a gene, promoter or allele originally found in a given plant or cell.

“Isolated nucleic acid”, used interchangeably with “isolated DNA” as used herein refers to a nucleic acid not occurring in its natural genomic context, irrespective of its length and sequence. Isolated DNA can, for example, refer to DNA which is physically separated from the genomic context, such as a fragment of genomic DNA. Isolated DNA can also be an artificially produced DNA, such as a chemically synthesized DNA, or such as DNA produced via amplification reactions, such as polymerase chain reaction (PCR) well-known in the art. Isolated DNA can further refer to DNA present in a context of DNA in which it does not occur naturally. For example, isolated DNA can refer to a piece of DNA present in a plasmid. Further, the isolated DNA can refer to a piece of DNA present in another chromosomal context than the context in which it occurs naturally, such as for example at another position in the genome than the natural position, in the genome of another species than the species in which it occurs naturally, or in an artificial chromosome.

Any of the nucleic acid sequences described above may be provided in a vector. A vector typically comprises, in a 5′ to 3′ orientation: a promoter to direct the transcription of a nucleic acid sequence and a nucleic acid sequence. The vector may further comprise a 3′ transcriptional terminator, a 3′ polyadenylation signal, other untranslated nucleic acid sequences, transit and targeting nucleic acid sequences, selectable markers, enhancers, and operators, as desired. The wording “5′ UTR” refers to the untranslated region of DNA upstream, or 5′ of the coding region of a gene and “3′ UTR” refers to the untranslated region of DNA downstream, or 3′ of the coding region of a gene. Means for preparing recombinant vectors are well known in the art. Methods for making vectors particularly suited to plant transformation are described in U.S. Pat. Nos. 4,971,908, 4,940,835, 4,769,061 and 4,757,011. Typical vectors useful for expression of nucleic acids in higher plants are well known in the art and include vectors derived from the tumor-inducing (Ti) plasmid of Agrobacterium tumefaciens. One or more additional promoters may also be provided in the recombinant vector. These promoters may be operably linked, for example, without limitation, to any of the nucleic acid sequences described above. Alternatively, the promoters may be operably linked to other nucleic acid sequences, such as those encoding transit peptides, selectable marker proteins, or antisense sequences. These additional promoters may be selected on the basis of the cell type into which the vector will be inserted. Also, promoters which function in bacteria, yeast, and plants are all well taught in the art. The additional promoters may also be selected on the basis of their regulatory features. Examples of such features include enhancement of transcriptional activity, inducibility, tissue specificity, and developmental stage-specificity.

The vector may also contain one or more additional nucleic acid sequences. These additional nucleic acid sequences may generally be any sequences suitable for use in a vector. Such nucleic acid sequences include, without limitation, any of the nucleic acid sequences, and modified forms thereof, described above. The additional structural nucleic acid sequences may also be operably linked to any of the above-described promoters. The one or more structural nucleic acid sequences may each be operably linked to separate promoters. Alternatively, the structural nucleic acid sequences may be operably linked to a single promoter (i.e. a single operon).

The invention further provides a plant cell, plant part or seed of the wheat plant according to the invention. A mutant allele of the above-described wheat EOD1 gene is also provided which may comprises the above specified mutations.

As used herein, the term “allele(s)” means any of one or more alternative forms of a gene at a particular locus. In a diploid (or amphidiploid) cell of an organism, alleles of a given gene are located at a specific location or locus (loci plural) on a chromosome. One allele is present on each chromosome of the pair of homologous chromosomes.

As used herein, the term “locus” (loci plural) means a specific place or places or a site on a chromosome where for example a gene or genetic marker is found. For example, the “EOD1 A locus” refers to the position on a chromosome of the A subgenome where a EOD1 A gene (and two EOD1 A alleles) may be found, while the “EOD1 B locus” refers to the position on a chromosome of the B subgenome where a EOD1 B gene (and two EOD1 B alleles) may be found and the “EOD1 D locus” refers to the position on a chromosome of the D genome where a EOD1 D gene (and two EOD1 D) alleles) may be found.

A “wild type allele” refers to an allele of a gene required to produce the wild-type protein and wild type phenotype. By contrast, a “mutant plant” refers to a plant with a different rare phenotype of such plant in the natural population or produced by human intervention, e.g. by mutagenesis or gene editing, and a “mutant allele” refers to an allele of a gene required to produce the mutant protein and/or the mutant phenotype and which is produced by human intervention such as mutagenesis or gene editing.

As used herein, the term “wild type EOD1” means a naturally occurring EOD1 allele found within wheat plant plants, which encodes a functional EOD1 protein. In contrast a “EOD1 mutant allele” refers to an allele which does not encode a functional EOD1 protein or encodes a EOD1 protein having a reduced activity compared to a functional EOD1 protein.

In yet another embodiment, a method of increasing yield of a wheat plant compared to a wild type or control wheat plant is provided comprising reducing or abolishing the expression of at least one EOD1 nucleic acid, as described herein, and/or reducing the activity of an EOD1 polypeptide, as described herein, in said plant. A method of producing a wheat plant with increased yield compared to a wild type or control wheat plant is also provided which comprises reducing or abolishing the expression of at least one EOD1 nucleic acid and/or reducing the activity of an EOD1 polypeptide in said plant.

These methods may comprise introducing at least one mutant allele according to the invention or at least one mutation in at least one nucleic acid sequence encoding EOD1 or at least one mutation in the promoter of at least one EOD1 gene in the cells of a wheat plant, as described above. These methods may comprise introducing or providing the silencing construct of the invention to cells of a wheat plant.

Introducing” in connection with the present application relates to the placing of genetic information in a plant cell or plant by artificial means. This can be done by any method known in the art for introducing RNA or DNA into plant cells, protoplasts, calli, roots, tubers, seeds, stems, leaves, seedlings, embryos, pollen and microspores, other plant tissues, or whole plants. “Introducing” also comprises stably integrating into the plant's genome. Introducing the construct can be performed by transformation or by crossing with a plant obtained by transformation or its descendant (also referred to as “introgression”). Introducing an allele also may be performed by mutagenesis of by gene editing.

The term “providing” may refer to introduction of a construct to a plant cell by transformation, optionally followed by regeneration of a plant from the transformed plant cell. The term may also refer to introduction of the construct by crossing of a plant comprising the construct with another plant and selecting progeny plants which have inherited the construct. Yet another alternative meaning of providing refers to introduction of the construct by techniques such as protoplast fusion, optionally followed by regeneration of a plant from the fused protoplasts.

The construct may be provided to a plant cell by methods well-known in the art.

The term “transformation” herein refers to the introduction (or transfer) of nucleic acid into a recipient host such as a plant or any plant parts or tissues including plant cells, protoplasts, calli, roots, tubers, seeds, stems, leaves, seedlings, embryos and pollen. Plants resulting from transformation are referred to as cisgenic, intragenic or transgenic plants, depending on the origin of the nucleic acid compared to the transformed plant species. Transformed, intragenic, cisgenic, transgenic and recombinant refer to a host organism such as a plant into which an isolated nucleic acid or a heterologous nucleic acid molecule (e.g. a recombinant gene or vector) has been introduced. The nucleic acid can be stably integrated into the genome of the plant.

The invention further provides a method for identifying and/or selecting a wheat plant having an increased yield to a wild type or control wheat plant comprising detecting in the plant at least one mutant allele of the invention or at least one mutation in at least one nucleic acid sequence encoding EOD1 or at least one mutation in the promoter of EOD1 resulting in a reduced level of EOD1 gene expression or abolished expression of at least one EOD1 nucleic acid and/or in a reduced activity of an EOD1 polypeptide in said plant compared to a wild type or control wheat plant.

Mutant alleles according to the invention or mutations in the nucleic acid sequence encoding EOD1 or in the promoter of EOD1 resulting in a reduced level of EOD1 gene expression or abolished expression of an EOD1 nucleic acid and/or in a reduced activity of an EOD1 polypeptide can be detected by molecular methods well known in the art, such as genotyping methods or sequencing.

Means and methods to determine the expression level of a given gene are well known in the art including, but not limited to, quantitative reverse transcription polymerase chain reaction (quantitative RT-PCR) for the detection and quantification of a specific mRNA and enzyme-linked immunosorbent assay (ELISA) for the detection and quantification of a specific protein. Means and methods to determine protein function are well known in the art including, but not limited to bioassays capable of quantification of enzymatic activity and in silico prediction of amino acid changes that affect protein function, as further described herein.

Further provided is the use of a mutant allele of the invention or a loss of function or partial loss of function mutation in at least one nucleic acid sequence encoding EOD1 or at least one mutation in the promoter of EOD1 or of an RNA interference construct that reduces or abolishes the expression of an EOD1 nucleic acid and/or reduces or abolishes the activity of an EOD1 promoter to increase yield of a wheat plant.

Lastly a method of producing food, feed, or an industrial product is provided which comprises (a) obtaining the wheat plant of the invention or a part thereof, and (b) preparing the food, feed or industrial product from the plant or part thereof. The food or feed may be meal, grain, starch, flour or protein. The industrial product may be biofuel, fiber, industrial chemicals, a pharmaceutical or a nutraceutical.

In case of a wheat plant or other cereal plant, examples of food products include flour, starch, leavened or unleavened breads, pasta, noodles, animal fodder, breakfast cereals, snack foods, cakes, malt, pastries, seitan and foods containing flour-based sauces.

Method of producing such food, feed or industrial product from wheat are well known in the art. For example, the flour is produced by grinding finely grains in a mill (see for example www.madehow.com/Volume-3/Flour.html) and the biofuel is produced from wheat straw or mixtures of wheat straw and wheat meal (see for example Erdei et al., Biotechnology for Biofuels, 2010, 3:16).

The plants according to the invention may additionally contain an endogenous or a transgene, which confers herbicide resistance, such as the bar or pat gene, which confer resistance to glufosinate ammonium (Liberty®, Basta® or Ignite®) [EP 0 242 236 and EP 0 242 246 incorporated by reference]; or any modified EPSPS gene, such as the 2mEPSPS gene from maize [EPO 508 909 and EP 0 507 698 incorporated by reference], or glyphosate acetyltransferase, or glyphosate oxidoreductase, which confer resistance to glyphosate (RoundupReady®), or bromoxynitril nitrilase to confer bromoxynitril tolerance, or any modified AHAS gene, which confers tolerance to sulfonylureas, imidazolinones, sulfonylaminocarbonyltriazolinones, triazolopyrimidines or pyrimidyl(oxy/thio)benzoates.

The plants or seeds of the plants according to the invention may be further treated with a chemical compound, such as a chemical compound selected from the following lists: Herbicides: Clethodim, Clopyralid, Diclofop, Ethametsulfuron, Fluazifop, Glufosinate, Glyphosate, Metazachlor, Quinmerac, Quizalofop, Tepraloxydim, Trifluralin. Fungicides/PGRs: Azoxystrobin, N-[9-(dichloromethylene)-1,2,3,4-tetrahydro-1,4-methanonaphthalen-5-yl]-3-(difluoromethyl)-1-methyl-1H-pyrazole-4-carboxamide (Benzovindiflupyr, Benzodiflupyr), Bixafen, Boscalid, Carbendazim, Carboxin, Chlormequat-chloride, Coniothryrium minitans, Cyproconazole, Cyprodinil, Difenoconazole, Dimethomorph, Dimoxystrobin, Epoxiconazole, Famoxadone, Fluazinam, Fludioxonil, Fluopicolide, Fluopyram, Fluoxastrobin, Fluquinconazole, Flusilazole, Fluthianil, Flutriafol, Fluxapyroxad, Iprodione, Isopyrazam, Mefenoxam, Mepiquat-chloride, Metalaxyl, Metconazole, Metominostrobin, Paclobutrazole, Penflufen, Penthiopyrad, Picoxystrobin, Prochloraz, Prothioconazole, Pyraclostrobin, Sedaxane, Tebuconazole, Tetraconazole, Thiophanate-methyl, Thiram, Triadimenol, Trifloxystrobin, Bacillus firmus, Bacillus firmus strain I-1582, Bacillus subtilis, Bacillus subtilis strain GB03, Bacillus subtilis strain QST 713, Bacillus pumulis, Bacillus. pumulis strain GB34. Insecticides: Acetamiprid, Aldicarb, Azadirachtin, Carbofuran, Chlorantraniliprole (Rynaxypyr), Clothianidin, Cyantraniliprole (Cyazypyr), (beta-) Cyfluthrin, gamma-Cyhalothrin, lambda-Cyhalothrin, Cypermethrin, Deltamethrin, Dimethoate, Dinetofuran, Ethiprole, Flonicamid, Flubendiamide, Fluensulfone, Fluopyram, Flupyradifurone, tau-Fluvalinate, Imicyafos, Imidacloprid, Metaflumizone, Methiocarb, Pymetrozine, Pyrifluquinazon, Spinetoram, Spinosad, Spirotetramate, Sulfoxaflor, Thiacloprid, Thiamethoxam, 1-(3-chloropyridin-2-yl)-N-[4-cyano-2-methyl-6-(methylcarbamoyl)phenyl]-3-[5-(trifluoromethyl)-2H-tetrazol-2-yl]methyl-1H-pyrazole-5-carboxamide, 1-(3-chloropyridin-2-yl)-N-[4-cyano-2-methyl-6-(methylcarbamoyl)phenyl]-3-[5-(trifluoromethyl)-1H-tetrazol-1-yl]methyl-1H-pyrazole-5-carboxamide, 1-2-fluoro-4-methyl-5-[(2,2,2-trifluorethyl) sulfinyl]phenyl-3-(trifluoromethyl)-1H-1,2,4-triazol-5-amine, (1E)-N-[(6-chloropyridin-3-yl)methyl]-N′-cyano-N-(2,2-difluoroethyl) ethanimidamide, Bacillus firmus, Bacillus firmus strain I-1582, Bacillus subtilis, Bacillus subtilis strain GB03, Bacillus subtilis strain QST 713, Metarhizium anisopliae F52.

The term “comprising” is to be interpreted as specifying the presence of the stated parts, steps or components, but does not exclude the presence of one or more additional parts, steps or components. A plant comprising a certain trait may thus comprise additional traits.

It is to be understood that this invention is not limited to the particular methodology, protocols, cell lines, plant species or genera, constructs, and reagents described as such. It is also to be understood that the terminology used herein is for the purpose of describing particular embodiments only and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth.

### SEQUENCE LISTING

The sequence listing contained in the file named “210428_SEQLISTING_Std26_v3.xml”, which is 46 kilobytes (size as measured in Microsoft Windows®), contains 26 sequences SEQ ID NO: 1 through SEQ ID NO: 26 is filed herewith by electronic submission and is incorporated by reference herein.

In the description and example, reference is made to the following sequences:
SEQ ID NO: 1: amino acid acid sequence of the protein EOD1 from the A subgenome
SEQ ID NO: 2: nucleotide sequence of the coding DNA sequence of EOD1 from the A subgenome
SEQ ID NO: 3: nucleotide sequence of the genomic DNA encoding EOD1 from the A subgenome
SEQ ID NO: 4: amino acid acid sequence of the protein EOD1 from the B subgenome
SEQ ID NO: 5: nucleotide sequence of the coding DNA sequence of EOD1 from the B subgenome
SEQ ID NO: 6: nucleotide sequence of the genomic DNA encoding EOD1 from the B subgenome
SEQ ID NO: 7: amino acid acid sequence of the protein EOD1 from the D subgenome
SEQ ID NO: 8: nucleotide sequence of the coding DNA sequence of EOD1 from the D subgenome
SEQ ID NO: 9: nucleotide sequence of the genomic DNA encoding EOD1 from the D subgenome
SEQ ID NO: 10: nucleotide sequence of the promoter of EOD1 from the A subgenome
SEQ ID NO: 11: nucleotide sequence of the promoter of EOD1 from the B subgenome
SEQ ID NO: 12: nucleotide sequence of the promoter of EOD1 from the D subgenome
SEQ ID NO: 13: nucleotide sequence of the primer specific for the detection of the wild type allele of the EOD1 gene from the A subgenome by KASP assay
SEQ ID NO: 14: nucleotide sequence of the of the FAM tail for the detection of the wild type allele of the EOD1 gene from the A subgenome by KASP assay
SEQ ID NO: 15: nucleotide sequence of the primer specific for the detection of the EOD1 A1 mutant allele of the EOD1 gene from the A subgenome
SEQ ID NO: 16: nucleotide sequence of the VIC tail for the detection of the EOD1 A1 mutant allele of the EOD1 gene from the A subgenome
SEQ ID NO: 17: nucleotide sequence of the common primer for the detection of both the wild type and the EOD1 A1 mutant allele of the EOD1 gene from the A subgenome
SEQ ID NO: 18: nucleotide sequence of the primer specific for the detection of the wild type allele of the EOD1 gene from the D subgenome by KASP assay
SEQ ID NO: 19: nucleotide sequence of the of the FAM tail for the detection of the wild type allele of the EOD1 gene from the D subgenome by KASP assay
SEQ ID NO: 20: nucleotide sequence of the primer specific for the detection of the EOD1 D3 mutant allele of the EOD1 gene from the D subgenome
SEQ ID NO: 21: nucleotide sequence of the VIC tail for the detection of the EOD1 D3 mutant allele of the EOD1 gene from the D subgenome
SEQ ID NO: 22: nucleotide sequence of the common primer for the detection of both the wild type and the EOD1 D3 mutant allele of the EOD1 gene from the D subgenome
SEQ ID NO: 23: nucleotide sequence of the forward primer to pre-amplify the allele EOD1 A1
SEQ ID NO: 24: nucleotide sequence of the reverse primer to pre-amplify the allele EOD1 A1
SEQ ID NO: 25: nucleotide sequence of the forward primer to pre-amplify the allele EOD1 D3
SEQ ID NO: 26: nucleotide sequence of the reverse primer to pre-amplify the allele EOD1 D3.

### EXAMPLES

### Example 1—Isolation of the DNA Sequences of the EOD1 Genes in Wheat

The EOD1 nucleotide sequences from Triticum aestivum have been determined as follows:

Three contigs containing wheat EOD1 genes were identified in the Chinese Spring survey sequence. The three contigs had the following identifiers: A02L6329354; B02L8057531; D02L9907683. All three contigs contained a complete wheat EOD1 gene consisting of 5 exons and 4 introns. The three wheat EOD1 genes are located on chromosomes A01, B01 and D01, respectively.

Later releases of the Chinese Spring reference genome confirmed the complete sequences of the three homoeologous EOD1 genes and enabled identification of the corresponding promoters of the three wheat EOD1 genes. The relevant sequences are further described below as SEQ ID Nos 1-12.

SEQ ID NOs: 3, 6 and 9 are the genomic sequences of TaEOD1 from the A subgenome, TaEOD1 from the B subgenome and Ta EOD1 from the D subgenome, respectively of T. aestivum. SEQ ID NOs: 2, 5 and 8 are the cDNA (coding) sequences of TaEOD1 from the A subgenome, TaEOD1 from the B subgenome and TaEOD1 from the D subgenome, respectively. SEQ ID Nos: 1, 4 and 7 are the amino acid sequences of the proteins encoded by TaEOD1 from the A subgenome, TaEOD1 from the B subgenome and TaEOD1 from the D subgenome, respectively. SEQ ID NOs: 10, 11 and 12 are the promoter sequences of TaEOD1 from the A subgenome, TaEOD1 and TaEOD1 from the D subgenome, respectively.

### Example 2—Generation and Isolation of Mutant for the Different EOD1 Genes in Wheat

Mutations in the EOD1 genes of Triticum aestivum identified in Example 1 were generated and identified as follows:
20,000 seeds from an elite spring wheat breeding line (M0 seeds) were pre-imbibed during 15 minutes in distilled water containing 10% Tween-20 and thoroughly rinsed by 4 wash steps. The seeds were subsequently exposed to 0.65% EMS (Sigma: M0880) and incubated on a rotary shaker during 16 hours.
The mutagenized seeds (M1 seeds) were rinsed three times and dried in a fume hood during 2 hours. 20,000 M1 seeds were planted and ca. 2000 surviving M1 plants were grown in soil and selfed to generate M2 seeds. M2 seeds were harvested for each individual M1 plant.
2000 M2 plants, one from each M2 seed lot, were grown and DNA samples were prepared from leaf samples of each individual M2 plant according to the CTAB method (Doyle and Doyle, 1987, Phytochemistry Bulletin 19:11-15).
The DNA samples were screened for the presence of point mutations in the three homoeologous EOD1 genes on chromosomes A03, B03 and D03, causing the introduction of STOP codons or amino acid changes in the protein-encoding sequence. For this purpose, the three wheat EOD1 genes were first amplified from all three subgenomes using homoeolog-specfic exon-spanning primers. In a next step consecutive and slightly overlapping regions of ca. 200-bp were amplified using nested and bar-coded primers. In a third step the 200-bp amplicons were pooled for construction of sequencing libraries and subjected to amplicon sequencing using Illumina Next Generation Sequencing techniques (KeyGene). In a final (validation) step the resulting sequences were analyzed for the presence of the point mutations in the EOD1 genes using dedicated software, such as the NovoSNP software (VIB Antwerp).

TABLE 2
summarizes the mutant alleles of wheat EOD1 genes that were identified.

| | | | | | Amino-acid- |
|---|---|---|---|---|---|
| Mutant | Contig | position | Effect | Mutation | change |
| A1 | A02L6329354 | 6004 | splice site | G-A | NA |
| D3 | D02L9907683 | 6652 | splice site | G-A | NA |

NA: not applicable.

Seeds of plants comprising mutant alleles of wheat EOD1 genes in homozygous state have been deposited at the NCIMB, Ferguson Building, Craibstone Estate, Bucksburn, Aberdeen, AB 21 9YA UK, under the Budapest Treaty on 20 Sep. 2021, under accession numbers NCIMB 43860 (EOD1 D3) and NCIMB 43861 (EOD1 A1).

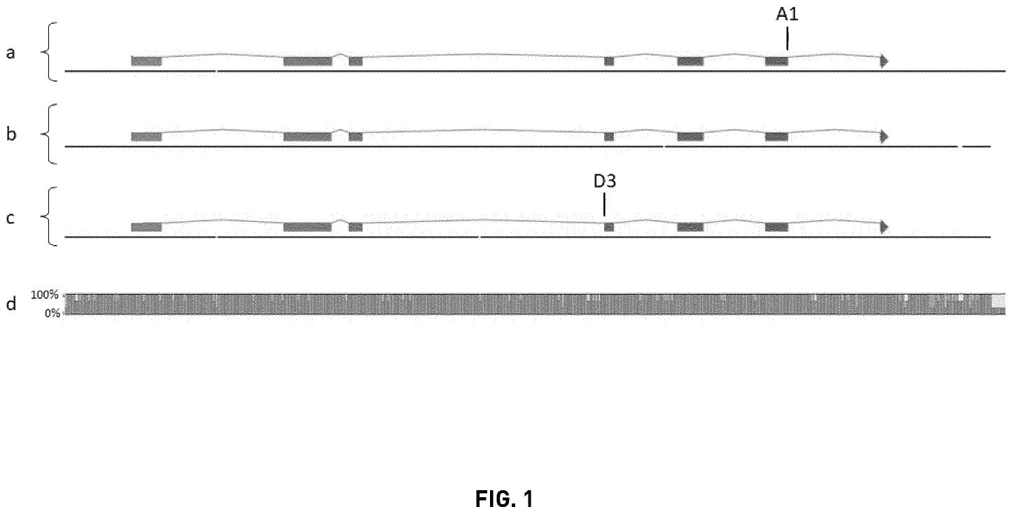

FIG. 1 visualizes the position of the selected and validated wheat EOD1 mutations on the annotated gene sequences.

### Example 3—Identification of a Triticum aestivum Plant Comprising Wheat Eod1 Mutant Alleles (Include Process for Backcrossing and Stacking, Introduce/Explain the Two Families of Eod1 Mutants)

Wheat plants comprising the mutations in the EOD1 genes identified in Example 2 were identified as follows:
For each mutant EOD1 gene identified in the DNA sample of an M2 plant, at least 50 M3 plants harvested from the M2 plant comprising the EOD1 mutation were grown and DNA samples were prepared from leaf samples of each individual M3 plant.
The DNA samples were screened for the presence of the identified EOD1 mutation by amplicon sequencing.
Heterozygous and homozygous (as determined based on the sequencing results) M3 plants comprising the expected EOD1 mutation were identified and used for seed production by selfing and cross-pollination.selfed and M3 seeds were harvested. Whenever possible homozygous plants were preferred for seed production.
In all subsequent generations wheat plants containing the intended EOD1 mutant genotype for crossing, backcrossing, or selfing were identified using dedicated genotyping assays, such as KASP markers (see Example 5).

Wheat plants containing combinations of mutations in one or two homoeologous EOD1 genes were obtained as follows:
An M3 plant containing mutation A1 has been crossed to an M3 plant containing mutation D3. In the progeny of this cross F1 plants were selected that were heterozygous for mutation A1 and D3. These F1 plants were intercrossed. In the intercross progeny plants were selected that were heterozygous for both mutations: (A1/-, D3/-). These plants were backcrossed to wild-type plants of the same cultivar during two generations, with selection of double heterozygous plants in each generation. After two backcrosses, the double heterozygous plants were selfed and in the progeny the following three homozygous genotypes were selected: (A1,-, D3); (A1,-,-); (-,-, D3). All homozygous genotypes occurred at a frequency of 1/16. Homozygous plants were further increased during several generations by self pollination to produce sufficient seed for field trial evaluation.

### Example 4—Analysis of Triticum aestivum Plants Comprising Wheat Eod1 Alleles Under Field Conditions

Wheat plants homozygous for mutations in EOD1 genes in one or two of the three subgenomes were grown under field conditions in various locations in Germany and France.

Field trials were performed in 10 locations and addressed yield performance across different environments.

TABLE 3
wheat lines tested.

| Genotype | Short name |
|---|---|
| double homozygous mutant eodl A1 eodl D3 | EOD1 (A1/—/D3) |
| single homozygous mutant eodl A1 | EOD1 (A1/—/—) |
| single homozygous mutant eodl D3 | EOD1 (—/—/D3) |
| wild type segregant | EOD1 (—/—/—) |

The “-” sign indicates the presence of the wildtype homozygous allele of the EOD1 gene in the different subgenomes.

TABLE 4
Measurements performed

| | | Observed | | |
|---|---|---|---|---|
| PRISM | Abbreviation | character | How? | Unit |
| Heading | HD | Days | Record the days past | Days |
| Date | | to | planting when 50% | after |
| | | heading | of main tillers show | planting |
| | | | whole ear | |
| Grain | YLDP | Yield per | Raw plot yield in | Grams |
| yield/plot | | Plot | grams per plot, not | per plot |
| | | | adjusted for moisture | |
| Grain | YLDHA | Grain | Calculate: yield per | t/ha |
| Yield | | Yield per | plot to tonne per ha | |
| tonne/ha | | hectare | and to 15% moisture | |
| | | | content | |
| Moisture | MOI | Moisture | Measured on | % |
| % | | | combine | |
| Kernels | YLDS | Grain | Calculated: | #kernels/ |
| per Plot | | number | 1000 s/TGW*YLDR | plot |
| Grain | TGW | Thousand | Determined by Agro- | gramm/ |
| weight | | grain | optie or Marvin | 1000 |
| | | weight | device | grains |

### Description of Data Analysis

Data were explored for quality, obvious outliers were removed and for each location the means by entry and the deltas between the mutants and their corresponding wild type segregants were calculated for each variable.

Further analysis of yield (YLDHA) and yield components (TKW and YLDS) is performed with mixed model and means were adjusted for spatial variance resulting in estimates by location. Based on the estimates by location, the contrasts of the mutants in percentage effect relative to the corresponding wild type segregant, including 95% confidence intervals (p<0.05), were generated by location. Based on the calculated means of the estimates by location, the contrasts of the mutants in percentage effect relative to the corresponding wild type segregant, including 95% confidence intervals (p<0.05), were generated across locations.

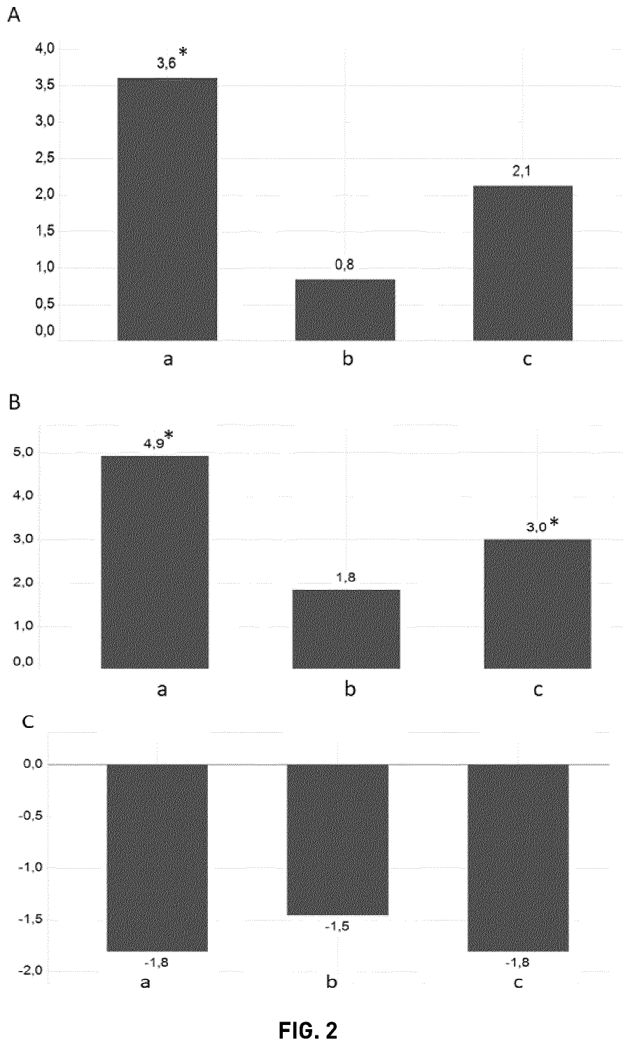

### Field Trial Results (FIG. 2)

From the 4 different genotypes tested, one genotype (EOD1 (A1/-/D3) showed a significant yield increase across all 10 testing locations. This contrast to the corresponding wildtype segregant was statistically significant with a p-value of <0.05.

For this improved genotype the increased yield performance resulted from an increase of grain weight (TGW) compared to the corresponding wildtype segregant.

One additional genotype (EOD1 (-/-/D3) showed an increase in yield compared to the corresponding wildtype segregant, however, this increase was not statistically significant. This line however showed a statistically significant increase in grain weight (TKW) compared to the corresponding wildtype segregant.

TABLE 5
overview of contrasts (in %) of eodl mutants vs the corresponding
wildtype segregant for Yield, TGW and Grain number (YLDS) across
all 10 field locations

| Short name | YLDHA (%) | TGW (%) | YLDS (%) |
|---|---|---|---|
| EOD1 (A1/—/D3) | 3.6* | 4.93* | 1.81 |
| EOD1 (A1/—/—) | 0.84 | 1.84 | −1.45 |
| EOD1 (—/—D3) | 2.12 | 3.01* | −1.81 |

(*significant change with p-value < 0.05).

### Example 5—Detection Method of the Mutant Alleles

To select for plants comprising a point mutation in a EOD1 allele, direct sequencing by standard sequencing techniques known in the art can be used. Alternatively, PCR based assays can be developed to discriminate plants comprising a specific point mutation in a EOD1 allele from plants not comprising that specific point mutation. The following KASP assays were developed to detect the presence or absence and the zygosity status of the mutant alleles identified in Example 2:

### Template DNA:

Genomic DNA isolated from leaf material of homozygous or heterozygous mutant wheat plants (comprising a mutant EOD1 allele).
Wild type DNA control: Genomic DNA isolated from leaf material of wild type wheat plants (comprising the wild type equivalent of the mutant EOD1 allele).
Positive DNA control: Genomic DNA isolated from leaf material of homozygous mutant wheat plants known to comprise.
Primers and probes for the mutant and corresponding wild type target EOD1 gene are indicated in Table 6.

TABLE 6
overview of the sequences used for the identification of the
different eod1 mutant alleles and EOD1 wild type alleles

| | | | | SEQ ID NO | | |
|---|---|---|---|---|---|---|
| | | SEQ ID NO | SEQ ID NO | of primer | SEQ ID NO | SEQ ID NO |
| Target | | of primer | of FAM | mutant | of VIC | of common |
| gene | mutant | WT allele | tail | allele | tail | primer |
| EOD1 | eod1 | 13 | 14 | 15 | 16 | 17 |
| A | A1 | | | | | |
| EOD1 | eod1 | 18 | 19 | 20 | 21 | 22 |
| D | D3 | | | | | |

Optionally the target sequences for each gene may be pre-amplified first by PCR using the primers having SEQ ID NO: 23 and 24 and SEQ ID NO: 25 and 26 respectively for the eod1 mutant alleles A1 and D3.

1. A wheat plant having a reduced level of EOD1 (ENHANCER OF DA1) gene expression and/or reduced activity of the EOD1 polypeptide compared to a wild type or a control plant.

2. The wheat plant of claim 1, wherein the EOD1 polypeptide comprises an amino acid sequence selected from the group consisting of:
a) the amino acid sequence of any one of SEQ ID NOs: 1, 4, 7; or
b) an amino acid sequence which comprises at least 80% sequence identity to any one of SEQ ID NOs: 1, 4, 7.

3. The wheat plant of claim 1, wherein the EOD1 nucleic acid comprises a nucleic acid sequence selected from the group consisting of:
a) the nucleic acid sequence of any one of SEQ ID NOs: 3, 6, 9 and SEQ ID NOs: 2, 5, 8;
b) a nucleic acid sequence having at least 80% sequence identity to any one of SEQ ID NOs: 3, 6, 9 and SEQ ID NOs: 2, 5, 8;
c) a nucleic acid sequence encoding the amino acid sequence of any one of SEQ ID NOs: 1, 4, 7; or
d) a nucleic acid sequence encoding an amino acid sequence which comprises at least 80% sequence identity to any one of SEQ ID NOs: 1, 4, 7.

4. The wheat plant of claim 1, wherein said plant is characterized by an increased yield compared to a wild-type or control pant.

5. The wheat plant of claim 4, wherein said increased yield is an increase in grain yield.

6. The wheat plant of claim 5, wherein said increase in grain yield is an increase in at least one of grain number and/or thousand grain weight.

7. The wheat plant of claim 1, wherein said plant comprises at least one mutation in at least one nucleic acid sequence encoding the EOD1 polypeptide or at least one mutation in the promoter of at least one of the EOD1 genes.

8. The wheat plant of claim 7, wherein said mutation is an insertion, deletion and/or substitution.

9. The wheat plant of claim 7, wherein said mutation is a loss of function or partial loss of function mutation.

10. The wheat plant of claim 9 wherein said mutation is selected from the group consisting of:
a) a G to A substitution at a position corresponding to position 3873 of SEQ ID NO: 3;
b) a G to A substitution at a position corresponding to position 2992 of SEQ ID NO: 9;
c) a G to A substitution at a position corresponding to position 3873 of SEQ ID NO: 3, and a G to A substitution at a position corresponding to position 2992 of SEQ ID NO: 9.

11. The wheat plant of claim 1, wherein said plant comprises a silencing construct that reduces or abolishes the expression of an EOD1 nucleic acid and/or reduces or abolishes the activity of an EOD1 promoter.

12. The wheat plant of claim 7, wherein the EOD1 promoter comprises the nucleic acid sequence of SEQ ID NOs: 10 to 12.

13. A plant cell, plant part or seed of the wheat plant according to claim 1.

14. A mutant allele of a wheat EOD1 gene, wherein the EOD1 gene is selected from the group consisting of:
a) a nucleic acid sequence comprising any one of SEQ ID NOs: 3, 6, 9;
b) a nucleic acid sequence comprising at least 80% sequence identity to any one of SEQ ID NOs: 3, 6, 9;
c) a nucleic acid sequence comprising the coding sequence of any one of SEQ ID NOs: 2, 5, 8;
d) a nucleic acid sequence comprising a coding sequence having at least 80% sequence identity to any one of SEQ ID NOs: 2, 5, 8;
e) a nucleic acid sequence encoding the amino acid sequence of any one of SEQ ID NOs: 1, 4, 7; or
f) a nucleic acid sequence encoding an amino acid sequence having at least 80% sequence identity with any one of SEQ ID NOs: 1, 4, 7.

15. The mutant allele according to claim 14, selected from the group consisting of:
a) a G to A substitution at a position corresponding to position 3873 of SEQ ID NO: 3;
b) a G to A substitution at a position corresponding to position 2992 of SEQ ID NO: 9.

16. A method of increasing yield of a wheat plant compared to a wild type or control wheat plant, said method comprising reducing or abolishing the expression of at least one EOD1 gene and/or reducing the activity of an EOD1 polypeptide in said plant.

17. A method of producing a wheat plant with increased yield compared to a wild type or control wheat plant, said method comprising reducing or abolishing the expression of at least one EOD1 gene and/or reducing the activity of an EOD1 polypeptide in said plant.

18. The method of claim 16, wherein the EOD1 polypeptide comprises an amino acid sequence selected from the group consisting of:
a) the amino acid sequence of SEQ ID NOs: 1, 4, 7; and
b) an amino acid sequence which comprises at least 80% sequence identity to SEQ ID NOs: 1, 4, 7.

19. The method of 16, wherein the EOD1 gene comprises a nucleic acid sequence selected from the group consisting of:
a) the nucleic acid sequence of any one of SEQ ID NOs: 3, 6, 9 or SEQ ID NOs: 2, 5, 8;
b) a nucleic acid sequence having at least 80% sequence identity to any one of SEQ ID NOs: 3, 6, 9 or SEQ ID NOs: 2, 5, 8;
c) a nucleic acid sequence encoding the amino acid sequence of any one of SEQ ID NOs: 1, 4, 7; and
d) a nucleic acid sequence encoding an amino acid sequence which comprises at least 80% sequence identity to any one of SEQ ID NOs: 1, 4, 7.

20. The method of claim 16, wherein said increased yield is an increase in grain yield.

21. The method of claim 20, wherein said increase in grain yield is an increase in at least one of grain number and/or thousand grain weight.

22. The method of claim 16, said method comprising introducing at least one mutant allele or at least one mutation in at least one nucleic acid sequence encoding EOD1 or at least one mutation in the promoter of at least one EOD1 gene.

23. The method of claim 22, wherein said mutation is an insertion, deletion and/or substitution.

24. The method of claim 22, wherein said mutation is a loss of function or partial loss of function mutation.

25. The method of claim 24, wherein said mutation is selected from the group consisting of:
a) a G to A substitution at a position corresponding to position 3873 of SEQ ID NO: 3;
b) a G to A substitution at a position corresponding to position 2992 of SEQ ID NO: 9;
c) a G to A substitution at a position corresponding to position 3873 of SEQ ID NO: 3, and a G to A substitution at a position corresponding to position 2992 of SEQ ID NO: 9.

26. The method of claim 16, the method comprising using a silencing construct that reduces or abolishes the expression of an EOD1 nucleic acid and/or reduces or abolishes the activity of an EOD1 promoter.

27. A wheat plant or wheat seed obtained by the method of claim 17.

28. A method for identifying and/or selecting a wheat plant having an increased yield compared to a wild type or control wheat plant, said method comprising detecting in the plant at least one mutant allele of a wheat EOD1 gene according to claim 14, at least one mutation in at least one nucleic acid sequence encoding EOD1 or at least one mutation in the promoter of at least one EOD1 gene resulting in a reduced level of EOD1 gene expression or abolished expression of at least one EOD1 gene and/or in a reduced activity of an EOD1 polypeptide in said plant compared to a wild type or control wheat plant.

29. The method of claim 28, wherein said mutation is an insertion, deletion and/or substitution.

30. The method of claim 28, wherein said mutation is a loss of function or partial loss of function mutation.

31. The method of claim 30, wherein said mutation is selected from the group consisting of:
a) a G to A substitution at a position corresponding to position 3873 of SEQ ID NO: 3;
b) a G to A substitution at a position corresponding to position 2992 of SEQ ID NO: 9;
c) a G to A substitution at a position corresponding to position 3873 of SEQ ID NO: 3, and a G to A substitution at a position corresponding to position 2992 of SEQ ID NO: 9.

32. (canceled)

33. (canceled)

34. (canceled)

35. A method of producing food, feed, or an industrial product comprising
a) obtaining the plant or a part thereof, of claim 1; and
b) preparing the food, feed or industrial product from the plant or part thereof.

36. The method of claim 35 wherein
a) the food or feed is meal, grain, starch, flour or protein; or
b) the industrial product is biofuel, fiber, industrial chemicals, a pharmaceutical or a nutraceutical.


SEQUENCE LISTING (26 sequences)

SEQ ID NO: 1 - protein, 253 aa, Triticum aestivum
MSMEVLHDTTGKKEAVVYYMDTPLPYAIEENFGGCFFEDDVDLAQVLQDQEIVYQLIQGNDGSGSSRTHSDPSSSCGHPRKSSRRRLKRGANYESQLAVDEALARDLQAMEDQIARATIDDKKRKEGRKPVSSSTSNNGNTSVSRSSQVLTEDGVDPDNMTYEELQQLGETIGTESKGLPEDVIALLKSSTYKIGIFSRKEKHDECVICCMAYKNRDKLTTLPCQHQYHRTCVAKWLKINKVCPVCNKEVFGS

SEQ ID NO: 2 - DNA, 762 bp, Triticum aestivum
  source 1..762: mol_type = unassigned DNA
atgagcatggaggtgctacacgatacaacagggaagaaggaagcggttgtgtactatatggacacccctttaccatatgcaattgaagagaatttcgggggatgcttctttgaagatgatgttgatcttgcacaagttcttcaagatcaggaaatagtgtatcaattaatccaaggaaatgatggaagtggttcatctaggactcattcagatcctagttctagttgtggccatccacgaaaatcgagtaggcgaagattaaaaagaggcgcaaattatgagtcacagctagctgtcgatgaagctttagctagagacttgcaggcgatggaggaccaaattgctagggcaacgatagatgacaagaagagaaaagaaggaagaaagcctgtaagttcttcaacatccaacaatggcaatacttctgttagcagatcttctcaggtattaacggaggatggagttgatcctgataatatgacttatgaggaactacaacaattaggggaaactattgggactgaaagtaaagggttgccagaagatgtgatagctctccttaagtcttcaacatacaaaattgggatattctcaagaaaggaaaaacatgatgaatgtgtcatatgctgtatggcttacaagaaccgcgataagctgactacattgccctgtcagcatcagtaccatcgaacttgtgttgccaaatggttgaagatcaataaggtatgccccgtttgcaacaaggaggtttttgggtcgtga

SEQ ID NO: 3 - DNA, 4797 bp, Triticum aestivum
  source 1..4797: mol_type = unassigned DNA
atatcagctgcgctccacacagcgaagcccggaggcgaaaaggggaccgaacaaggcggcctccggaccgcccgtcgatcctcccctctcactgggcacggcagcccccctcgcatcctccgaaatcccccattccccggttcgttcctaggtgagcttccacttccactcctgcacattcgctggttctgtttcgttgcgcggatgaaaatgtccattttttctgggaaagaaatggttgtcaggcgcttgatctgtggctcatggttgcttagatccgaggcattgggggcatcgactggtgggatggaacccctcttgcattccgcccatcgcttgtctgtccgtccgtccgtctgtgtggtttctcgaagagggattcagaatcacacacactgctttcctgtagtttagtactgtactttgtgttggtgatattaactacggctgctccggattttccccaatataagggtttcagttatcatgtcaagatgcaagggggtttcacttgctctaatagtcgatcgccgagaagcctgagataatccgatggttgaaagtacagtagtaaattccttgttattcagttggtacaacaatgattatgggtctgacagtaagatgcaacactaagtgggtatagtatgactagcagcgttctctgcttgcgcaaatctagcatgatccggcaatgttctacctgttccctttgtcaggttctcatgagcatggaggtgctacacgatacaacagggaagaaggaagcggttgtgtactatatggacacccctttaccatatgcaattgaagagaatttcgggggatgcttctttgaagatgatgttgatcttgcacaagttcttcaagatcaggtttcgtgccgttccctgttcatatcttatttggctgtgtccatcatgtcctggcttgcgtgttgttgtcgttgctcctgttagtttgcattagaaacctcagtcagttattctatcctagacatttgtccgttggggagggtatgatgctgcagttttctgttgtcttgtgcatagtaatctcagccaaatgtttttctatccaagggccaaagctgttcattgttttatgtcaagcttgaccaggtctagtaatatgatgtagaccatttctagtggatcatattggtacttaggtagttatcttaaatcacacaccataaaaatgattcatctttcatggcatttacgatccaagggccaaagcttttcatgacatttacactgcaaaattacaagctaattgcttgtcgccagatccaaatgggtcattggttcaccaaatgaccaaatatgtggaatcttcatttgaagtgtttaggcggaattgatatgtttttcttgtttgtagcacaaacagtgtagtacaaaatatgacagaaaattctcattaatttgcctattgtcacactctgttcaggaaatagtgtatcaattaatccaaggaaatgatggaagtggttcatctaggactcattcagatcctagttctagttgtggccatccacgaaaatcgagtaggcgaagattaaaaagaggcgcaaattatgagtcacagctagctgtcgatgaagctttagctagagacttgcaggcgatggaggaccaaattgctagggcaacgatagatgacaagaagagaaaagaaggtgattagttgtctgctggtactcttctatatcttgcaactcaatatggtacgacttttaaatgttgttaaaacacctgtttaggaagaaagcctgtaagttcttcaacatccaacaatggcaatacttctgttagcagatcttctcaggtatttactttcgacagctttgattctacagatttgcttcaaataatctattatataactaaaacaatgaagataaagccaccttgttattccacataggctaaattattggttcgttaatatatggtgggtcctaattataacggaggagatttaaaatctaacatcaaagctacatgcgtacatgtgttcgcatccaaacactaaagctaatgtccgacttggccagaagtattcagatcgtatcggagttgtattggctgatgtcatgaaataatcaaatgggaaatctttttgctttgtatatgcttcacacggatcatgactaaggtatatcatatttgtgtggcctctggctcggcaagacagacgtggtggcaattttcaaatttgcattagtttactttagctgtacacctttattagtggagaaaaacatagatctatctacgcataaagtctggaacaaaaagtaaaatagttatatttatctgatttatctgtgaattcaacgtaaaactaaaatgtggaataaaaataaaatacaactaaggaacaaacaatacctacgaccctcaacacgcttcgttcttacacagatgacatcctagaatacatgcagtaaaaaaattctaactctgggtatacataaaaaataatatttacattagtcaagtaaaaatactacctcctccttccatgtatacatggccaactagtcttgtctcaaatcagacctgtttaagtttgataagttcatagaaaaatatcagcatctacaatgccaaatcaatatcaccatatctattatgaagtatgttttcgtaatatattcattgggtgctgtagatgcggatgtatttttcctacaatcttggtcaaatttgaataattttaacttgagaaaagattaattggccttgcataaaatggaaggaaggagtatcattttgcgcagatcttctttcataatactacacttacagttttataatgtgcattcatatgaaatatgatcatcaatgttgcgtgctgaagaccgtgaaaattaaggcgtgaaagagtattaacaccataatgaaaatttagcgacgcaaatattgtgccgcacctgctagttatattttatttaatatgtgctttcctctatcttgccactgtaggtattaacggaggatggagttgatcctgataatatgacttatgaggtgtgtagcatcaagtgctttgtgtggccagtatattccctaatgttccatcaagggttgtgccccactaccagttaaatttttggttccctgatatattatctctgaaaggactcttgtagtctttgatgtgaatcgtggataacaagtgtatagatattatcttgtttatagtaagtagtacgaaaaaataaaatatacatccgtttgtttcgacatctattaagctatgttctatatacataaaaatgggatactacatgctatacctcttcaatttttgaatcacttttgtattacaggaactacaacaattaggggaaactattgggactgaaagtaaagggttgccagaagatgtgatagctctccttaagtcttcaacatacaaaattgggatattctcaagaaaggaaaaacatgatgagtatgtcctcaaaccgtcacttttttattgaacatgtgatataaatttgtgcataattgtcatggacagctatgcacaagccactcgccattttaccccaagaacctgcttcactgattaatctattgagtagtatctttataatcttaaggttcatcatatgacattgtgccagcatgtgatgttcctaaggtggcgtactattttctatgtatgctgtaaatgctcatatcaatccatattttaagtagcaaactatataaatatcctcttctgttaatgtttaaacaaacagatgtgtcatatgctgtatggcttacaagaaccgcgataagctgactacattgccctgtcagcatcagtaccatcgaacttgtgttgccaaatggttgaagatcaataaggtaagctaccttgcattcgcagcttttgtcattgatgagtattgcttccacaggaacttctagggattgttgtagttttctgtgagtgtgcgcagattgctacttgccatgtatttgtttcctggatctttgttcattgttaagttcagtcaggcgttagctcagaaccctttgtgtacttcctgatatgtatcaatgtatgtcaagtgcggtttatttttcgtcccgataggaagggaagttacggctagaaatctcgtagatatagtttagaacaaatgatggtgctggaagaaacagctcacctgattgttcatgataggggaggggatgtcttgtattgcttgctgttggtagtatgaccgttttcgtttactgtattgtctcttaagatgtttggcgtctgaccagcctagttcaattcgttgtgctgcgttgacaggtatgccccgtttgcaacaaggaggtttttgggtcgtgacagcaaagaccgtagattcacccgacggccaatgccgacatgcttaagaaacgttgtcgctcacgcaggccgcgaaaaaggcaacgcaatgtactctttagttatcgtataagatgaactggagcttctgttgcatcaccagcaaaataaagaaggtagatgcacatgttgagtttagcactttagctgttattatgtgacttggagtagtaatggatttcccacgggttccaagcttggcgcctcactgtatcgcaccatactgtagattgtgagtacccgtgacctattccctgaaaaaagaaaaagtgtagtggtgaccgttggcatgcttcctctgggttgcctctcagtattgtcttgttccatggcttggattggataaataaatctatggaatcggaccttgtctgggcagttaagattttctggattttgacttct

SEQ ID NO: 4 - protein, 253 aa, Triticum aestivum
MSMEVLHDTTGKKEAVVYYMDAPLPYAIEENFGGCFFEDDVDLAQVLQDQEIVYQLIQGNDGSGSSRTHSDPSSSCGHPRKSSGRRLTKGVNYESQLAVDEALARDLQAMEDQIAKATIDDKKRKEGRKPVSSSTSNNGNTSVSRSSQVLTEDGVDPDNMTYEELQQLGETIGTESKGLPEDVIALLKSSTYKIGIFSRKEKHDECVICCMAYKNRDKLTTLPCQHQYHRTCVAKWLKINKVCPVCNKEVFGS

SEQ ID NO: 5 - DNA, 762 bp, Triticum aestivum
  source 1..762: mol_type = unassigned DNA
atgagcatggaggtgctacatgatacaacagggaagaaggaagcggttgtgtactatatggatgcccccttgccatatgcaattgaagagaatttcgggggatgcttctttgaagatgatgttgatcttgcacaagttctccaagatcaggaaatagtgtatcaattaatccaaggaaatgatggaagtggttcatctaggactcattcagatcctagttctagttgtggccatccgcgaaaatcgagtgggcgaagattaacaaaaggcgtaaattatgagtcacagctagctgtcgatgaagctttagctagagacttgcaggcgatggaggaccaaatcgccaaggcaacaatagatgacaagaagagaaaagaaggaagaaagcccgtaagttcttcaacatccaacaatggcaatacttctgtgagcagatcttctcaggtattaacggaggatggagttgatcctgataatatgacttatgaggaactacaacaattaggggaaactattgggactgaaagtaaagggttgccagaagatgtgatagctctccttaagtcttcaacatacaaaattgggatattctcaagaaaggaaaaacatgatgaatgtgtgatatgctgtatggcttacaagaaccgcgataagctgactacattgccctgtcagcatcagtaccatcgaacttgtgttgccaaatggttgaagatcaataaggtatgccccgtttgcaacaaggaggtttttgggtcgtga

SEQ ID NO: 6 - DNA, 4789 bp, Triticum aestivum
  source 1..4789: mol_type = unassigned DNA
gagagaccaccatgatttgattctctctccccgactatgatatcagctgcgctccacacagcgaagcccggaggcgaaaagggggccgagcaaggcggcttgtggaccgcccgtcgatcctcttttcactcggcagtccccctcgcatcctccaaaatctcccattccccggttcgttcctaggtgagtctccactttccactcccatgctcgttgtcgcagattcgccggttctgtttcgtttcgcgaatgaagatgtccattttttgggagttgtcaggcgcttgatctgtgggctcgtgggggagatatcctagctgtttagatccgaggcattatgggcatcgatttctgcccatggcttgtctgtccgcctgtctctcgaggatggattcagagtcacagacactgccttcctgttgtttcatttagtattactagtgtcggtgatattaattacggttgctccggatttccccccagcaaaagggtttcaattatcatttcaagatgcaagggggtttcacttgctgtaacaatcgaccgccgagaagcctgagataatgtgatgtctgaaagcacactagtaaattccttgttgttcagttggtacaacaatgattgtgggtctgacaataagatgcaacaataagtgggtatagtacgattagcagcgttttctgcttgcgcaaatctagcatgatccggcaatgttctacctgttccctttgtcaggttctcatgagcatggaggtgctacatgatacaacagggaagaaggaagcggttgtgtactatatggatgcccccttgccatatgcaattgaagagaatttcgggggatgcttctttgaagatgatgttgatcttgcacaagttctccaagatcaggtttcgtgccgttccctgtccgcatcttatttggctgtgtccatcgtgtcttgacctgcgtgttgttgtcgttactcctgttatgcattagaaatctcagtcagatgttctatcctagacatttgtccgttggggagggtatgatgctgcagttttctgttgtcttgtgcatagtaatctcaaccaaatgtttttctatccaagggccaaagctgtttattttttttatgtcaagcttgaccaggtctagtaatatgagtatatgacgtagaccatttctagcggatcatatttgtactatgtagttatcttaaatcacacgccataaaaatgattcatctttcatgacatttatgatcgaagggccaaagcttttcatgacatttacgctgcaaaattacaaactaattgcttgtcgccagatccaaatgggtcatggttcaccaaatgaccaaatatgtggaatcttcatttaaagtgtttaggcggaattgatatatttttcttgtttgtagcacaaacagtgtagtacaaaatatgacagaacattctcattaatttgcctattgtcacactctgttcaggaaatagtgtatcaattaatccaaggaaatgatggaagtggttcatctaggactcattcagatcctagttctagttgtggccatccgcgaaaatcgagtgggcgaagattaacaaaaggcgtaaattatgagtcacagctagctgtcgatgaagctttagctagagacttgcaggcgatggaggaccaaatcgccaaggcaacaatagatgacaagaagagaaaagaaggtgattagttgtctgctggtactcttctataacttgcaactcaatatggtacgccttttaaatgttgccaacacctgtttaggaagaaagcccgtaagttcttcaacatccaacaatggcaatacttctgtgagcagatcttctcaggtatttactttcgacagctttgattctacagatttgcttcggataatctattatataactaaaacaatgaagataaagccaccttgttattccacgtaggctaaattatttggtccgttaagtgggtcctaattataaccgaggagatttaaaatctaacatcaaagctacatgcgtacatgtgtttgcatccaaacattaaagctaatgtccgacttggccagaagtattcagatcgtatcggagttgtattggctgatgtcatgaaataatcaaatgggaaatctttttgctttgtatatgcttcacatggatcatgactaaggtaaatcatatttgtgtggcctctggctcggcaagacagacgtggtggcaattttcaaatttgcatttgtttactttagctgtacaactttattagtggagaaaaacatagatctatctacgcataaagtctggaataaaaagtaaaatagttatatttatctgatttatctgtgaattcaacgtaaaactaaaatgtggaataaaaataaaatacaactaaggaacaaagaatacctacgaccctcaacacgcttcgttcttacacagatgacatcctagaatacatgcagtaaaacttttctaactctgggtatactaaaaaataatatttacattagtcaagtaaaaatactacctcctccttccatgtatacatggccaactagtcttgtctcaaatcagacctgtttaagtttgataagttcatagaaaaatatcagcatctacaatgccaaataaatatcaccatatctattatgaagtatgttttcgtaatatattcattgggtgttgtagatgcggatatatttttcctactatcttggtcaaatttgaataattttaacttcagaaaagattaattggccttgcataaaatggaaggaaggagtatcattttgcgcggatcttttttcataatactgcatttacagttttataatgtgtattcatatgaaatacgatcatcaatgttgtgtgctgaagaccgtgaaaattaaggcgtgtaagagtataacaccatataaaatttagcgacgcaaatatgtggcgcacctgctagttatattttatttaatgtgttttcctctatcttgccactgtaggtattaacggaggatggagttgatcctgataatatgacttatgaggtgtgtagcatcaagtgctttgtgtggccagtatattccctaatgttccatcaagggttgtgccccactaccagttaacttttggttccctgatatattatctttgaaaggactcttgtagtctttgatgtgaatcgtggataacaattgtatagatattatcttgtttatagtaagtagtacgaaaaaacataatacacatccgcttgtttcgacatctgttaagctatgctctaaaaatgggatgctacatgctatacctcttacttcaatttttgaatcacttgtgtattacaggaactacaacaattaggggaaactattgggactgaaagtaaagggttgccagaagatgtgatagctctccttaagtcttcaacatacaaaattgggatattctcaagaaaggaaaaacatgatgagtatgtcctcaagcctcgttcacttttttgttgaacatgtgatataaatttgtgcataattgtcatggacagctatgcacaaatcactcaccattttgtcccaagaacctgcttcactgattagtctattgagtagtatctttgtaatcttaaggttcatcatatgacattgtgccagcatgtgatgttcctagggtggagtactagtttctatgtatgttgtatatgctcatatcaatccatactttaagtaacaaactatataaatatcctcttctgttaatgtttaaacaaacagatgtgtgatatgctgtatggcttacaagaaccgcgataagctgactacattgccctgtcagcatcagtaccatcgaacttgtgttgccaaatggttgaagatcaataaggtaagctaccttgcctttgcagcttttgtaattgatgcgtattgcttccacaagaacttctagggattgttgtagtttctgtgagcgtgaccagattgctacttcccatataattgtttcttgcatctttattcattgttaaattcaatcagacgttagctcagaatcctttgtctacttcctgatatgtatcagtgtatgccaagtgcagtttattctccgtcctgataggaagggaagttatggctagaaatcttgttgatatagtttagagcaaatgatggcgctggaagaaacagctcatctaattgttaatgataggggagcgggtgtcctgtaatgcttgctgtcgacagtatgaccgttttccttcgctgttttttttctcttgagatgtttgacttctgatcagcttagttcaattcattgtgctgcgttgacaggtatgccccgtttgcaacaaggaggtttttgggtcgtgacagcaaagaccgtagattcacccgacggccaatgtcaacatgcttcagaaaacgttgccacttacgtgggcagcgaaaaaggcaacatgatgtactctttagttatcgtaagatgaaccggagcttctgttgcatcgccagcaaaataaagaaggtagatgctcgtgttgagtttagcactttagctgttatgtgattttttttttttttgcggggaagctgttatgtgacttggagtaatgtatttcccacgggtttcaagcttggcgcctcactgtattgaaccatactactgtatagattgtgagtagtcgtgacctattccctcaaaaaagaaaaggtgtagtggtgacctctgagtgttgtctttttccatggcttggatgaatagagctctgatggaaccggacctggtctgggttgttaagatt

SEQ ID NO: 7 - protein, 253 aa, Triticum aestivum
MSMEVLHDTTGKKEAVVYYMDAPLPYAIEENFGGCFFEDDVDLAQVLQDQEIVYQLIQGNDGSGLSRTHSDPGSSCGHPQKSSGRRLTRGANYESQLAVDEALARDLQAMEDQIARATIDDKKRKEGRKPVSSSTSNNGNTSVSRSSQVLTKDEVDPDNMTYEELQQLGETIGTESKGLPEDVIALLKSSTYKVGIFSRKEKHDECVICCMAYKNRDKLTTLPCQHQYHRTCVAKWLKINKVCPVCNKEVFGS

SEQ ID NO: 8 - DNA, 762 bp, Triticum aestivum
  source 1..762: mol_type = unassigned DNA
atgagcatggaggtgctacatgatacaacagggaagaaggaagcggttgtgtactatatggatgcccccttgccatatgcaattgaagagaatttcgggggatgcttctttgaagatgatgttgatcttgcacaagttcttcaagatcaggaaatagtgtatcaattaatccaaggaaatgatggaagtggtttatccaggactcattcagatcctggttctagttgtggccatccacaaaaatcgagtgggcgaagattaacaagaggcgcaaattatgagtcacagctagctgtcgatgaagctttagctagagacttgcaggcgatggaggaccaaattgctagggcaacgatagatgacaagaagagaaaagaaggaagaaagcccgtaagttcttcaacatccaacaatggcaatacttctgtgagcagatcttctcaggtattaacgaaggatgaagttgatcctgataatatgacttatgaggaactacaacaattaggggaaactattgggactgaaagtaaagggttgccagaagatgtgatagctctccttaagtcttcaacatacaaagttgggatattctcaagaaaggaaaaacatgatgaatgtgtgatatgctgtatggcttacaagaaccgcgataagctgactacattgccctgtcagcatcagtaccatcgaacttgtgttgccaaatggttgaagatcaacaaggtatgccccgtttgcaacaaggaggtttttgggtcgtga

SEQ ID NO: 9 - DNA, 4690 bp, Triticum aestivum
  source 1..4690: mol_type = unassigned DNA
gagagaccaccatgatttgattctctctcccccgactatgatatcagctgtgcgctccacacagcgaagcccggaggcgaaaaggggaccgagcaaggcggctcccggaccgcccgtcgatcctcccctctcattcggcagcccccctcgcatcctccgaaatcccccattccccggttcgttcctaggtgagcttctacttccactcctgcagattcgctggttctgtttcgtttcgcggatgaaaatgtccattttttctggcaaagaaatggttgtcaggcgcttgatctgtggctggtgggggagagatcctagttctttagatccgaggcattatggcatcgattgctgcccatggcttgtctgtccgcctgtctctcgaggatggattcaaactcaggcactgctttcctgtagtttagtatactagtgtcggtgatgttaattacggttgctccggatttccccccagcaaaagggtttcaattatcatttcaagatgcaagggggtttcacttgctgtaacagtcgaccgccgagaaccctgagataatgtgatgtctgaaagtacactagtaaattccttgttattcagttggtagaacaatgattgtgggtctgacaataagatgcaacactaagtggctatagtatgattagcagcgttttctgcttgcgcaaatctagcatgatccggtaatgttctacctgttcccttgtcaggttctcatgagcatggaggtgctacatgatacaacagggaagaaggaagcggttgtgtactatatggatgcccccttgccatatgcaattgaagagaatttcgggggatgcttctttgaagatgatgttgatcttgcacaagttcttcaagatcaggtttcatgccgttccctgtccgcatcttatttggctgtgtccatcgtgtcctagtctgcgtgttgttgtcgttgctcctgttagtttgcattagaaacctcagtcagatattctatcctagacatttgtccgttggggagggtatgatgctgcagttttctgttgtcttgtgcatagtaatctcaaccaaatgtttttctatccaagggccaaagctgttcatttttttatgtcaagcttgaccaggtgtagtaatatgatgtagaccatttctagcggatcatatttgtacttatgtagttatcttaaatcacacaccataaaaatgattcatctttcatgacatttatgatccaagggccaaagcttttcatgacatttacactgcaaaattacaaactaattgtttgtcgccagatccaaatgggttattggttcaccaaatgaccaaatatgtggaatcttcatttaaagtgtttaggcgtaattgatatgcttttcttgtttgtagcacaaacagcgtagtacaaaatatgacagaacattctcattaatttgcctattgtcaccctctgttcaggaaatagtgtatcaattaatccaaggaaatgatggaagtggtttatccaggactcattcagatcctggttctagttgtggccatccacaaaaatcgagtgggcgaagattaacaagaggcgcaaattatgagtcacagctagctgtcgatgaagctttagctagagacttgcaggcgatggaggaccaaattgctagggcaacgatagatgacaagaagagaaaagaaggtgattagttatctgctggtactcttctatatcttgcaactcaatatggtacgacttttaaatgttgttaagacacctgtttaggaagaaagcccgtaagttcttcaacatccaacaatggcaatacttctgtgagcagatcttctcaggtatttactttcgacagctttgattctacagatttgcttcgaataatctattatataactaaaacaatgaagataaagccaccttgttattccaaataggctaaattatttggttcgttaatatatgttgggtcctaattataacggaggagatttaaaatctaacatcaaagctacatgcgcacatgtgtttgcatccaaacattaaagttaatgtccgacttggcaagaagtatttcagatcgtatcggggttgtattggctgatgtcatgaaataatcaaatgagaaatctttttgctttgtatatgcttcacacggatcatgactaaggtaaatcatatttgtgtggcctctggctcggcaagacagatgtggtggcaattttcaactttgcattagtttactttagctgtacaactttattagtggagaaaaacatagatctatctacgcataaagtctggaataaaaagtaaaatagttatatttatctgatttatatgtgaattcaacgtaaaactaaaacgtggaataaaaataaaatacaactagggaacctacgaccctcaacacgcttcgttcttacacaaatgacatcctagaatacatgcagtaaaacttttctaactctgggtatacataaaaaataatatttacattgtcaagtaaaaataatacctcctccttccatatatacatggccaactagtcttgtctcaaatcagacctgtttaagtttgacaagtttatagaaaaatatcagcatctacaataccaaatcaatatcaccataattattatgaagtatgttttcataatatattcattgggtgttgtagatgcggatatatttttcgtacaattggtcaaatttaaataattttaacttgagaaaagattaattggccttgcataaaatggaaggaacgagtatcattttgcgcggatatttttttcgtaatactacatttacagttgtataatgtgtattcatatgaattatgatcatcaatgttgtgtgctgaaggccgtgaaaattaaggcatgtaagagtataacaccatattgaaaatctagcgacgcaaatatttggggcacctgctagttgtatttaatttaatatgtgttttcctctatcttgccactgcaggtattaacgaaggatgaagttgatcctgataatatgacttatgaggtgtgtagcatcaagtgctttgtgtgaccagtatattccctaatgttccatcaagggttgtgcctcactaccagttaatttttggttccctgatatattatatctgaaaggacccttgtagcctttgatgtgaatcgtggataacaattgtatagatattttcttgtttatagtaagtagtacgaaaaaataaaacacacatcagtttgtttcgacatctgttaagcaatgttctatatacataaaaatgggatgctacatgatatacctcttacttcaatttttgaatcacttgtgtattacaggaactacaacaattaggggaaactattgggactgaaagtaaagggttgccagaagatgtgatagctctccttaagtcttcaacatacaaagttgggatattctcaagaaaggaaaaacatgatgagtatgtcctcaagcctcgttcacttttttgttgaacttgtgatataaatttgtgcataattgtcatggacagctatgcacaagtcactcaccattttaccccaagaacctgcttcactgattaatctattgagtagtatctttgtaatcttaaggttcatcatatgacattgtgccagcatgtgatattcctatggtggagtactattttctatgtatgctgtatatgctcatatcaatccatattttaagtagcaaacaatttaaatgtcctcttctgttaatgtttaaacaaacagatgtgtgatatgctgtatggcttacaagaaccgcgataagctgactacattgccctgtcagcatcagtaccatcgaacttgtgttgccaaatggttgaagatcaacaaggtaagctaccttgcctttgcagcttttgtcattgatgcgtattgcttccacaggaacttctaggggttgttgtagttttctgtgaacatgaccagattcctacttgccatataattgtttcttggatctttattcattgttaaattcagtcagacgttagctcagaatcctttgtgtacttcctgatatgtatcaatgtatggcaagtgcagtttattcttcgtcctgataggaagggattttatggctagaaatgttgtagatatagtttagagcaaatgatggtgctggaagaaacagctcacctaattgttaatgataggggagtggatgtcctgtaatgcttgctgtcgacagtatgaccgttttccttcgctgttttctttctcttgagatgtttgacttctgatcagcttagttcaattcattgtgctgcgttgacaggtatgccccgtttgcaacaaggaggtttttgggtcgtgacagcgaagaccgtagattcacccgacgtccaatgtcaacatgcttcagaaacgctgtcacttacgcaggcaggccgcgaaagaggcaaactggatgtactctttagttatcgtataagatgaactggagcttctgttgcatcgccagcaaaataaagaaggcagatgctcgtgttgagtttagcactttagctgttatgtgacttggagtaatgtatttcccacgggtttcaagcttggcgcctttctgtattgcaccatactgtagattgtgtgtagtcgtgacctattccctcaaaaaagaaaaagggtagtggtgaccgttggcatgctt

SEQ ID NO: 10 - DNA, 2010 bp, Triticum aestivum
  source 1..2010: mol_type = unassigned DNA
ctgtaagtcacatcccatatgtctaggattcggtgtgtgagaaatgtcgtcagagttaagctatttgttggtttgatcatgaaaacagaaatgatgcctggaaacagatcattttccaccccacaaacttgcatcgggaaaagaaaaaggagcgcggaggaaaaaactgcaggtttattcgactcaattgggaatcaaactcaggctgccaatttgaatgccacacacgttaaccactaggacaagctcactttgttgattattagaggaaacaacgttatttggcccttcttttaatacaaacattaaacatatagtagtattatatgccagctataaattatttgaataaagaaaatagaaatttgaaaaagcattcacaaaaaccacgaattgaaagaaatcgaaattttgaaaaaggtttgtaccgattgaaaaaaattcatctattttgaaaaaaagttcatcgaatttgaatcaagttcatcgactctaaaaaaatgctcatcaatttgaaaaaaagttcatagactctggaaaaagactttaccaattttaaaaaagttcattaaattggaaacaaaaatcaaagattttgagaaaaagttcatattttttccaaaactgtcatcaaatttgaaaaaatcaacaattttgagaaaaaattcaccaaatttgaaaaaaacttcatctagtatgaaaaaggttcatcaaatttgaaaaaaaaaatcatcaacagttgaaaaacaattcatcaacttcgcagaaaaagctcatcgaaccaggaagaagaaaaaaaaaggaagagagaaaaaaatagaagtactaaaagaatgagaaagatgaagaatatcagacgaggttgcatggcatggtgggtagtccattacgggtcgaactctccccgcacacacttctttttgagtgtttggaaaccgaagagaagaacctactccctccgtccagaaatatttatcattaaaattgataaaagaagatgtatctagacatattttaattctagatacatccttttcatccattttgatgacaaatagtattttcgaacggaaggaatacatattaacacagataaatgggccggccagctaacgcggctgcaggcgccggttcgaatagggtttgcccctaatccaaaaggtgctggcagccgctgacgtcacgcctgcttccgggaatgatggccgcatataatttctttctgtactgtagcctgcagggtggcccatggcatgcgcgcacgcgtcccctcccagccactatgatatcagctgcgctccacacagcgaagcccggaggcgaaaaggggaccgaacaaggcggcctccggaccgcccgtcgatcctcccctctcactgggcacggcagcccccctcgcatcctccgaaatcccccattccccggttcgttcctaggtgagcttccacttccactcctgcacattcgctggttctgtttcgttgcgcggatgaaaatgtccattttttctgggaaagaaatggttgtcaggcgcttgatctgtggctcatggttgcttagatccgaggcattgggggcatcgactggtgggatggaacccctcttgcattccgcccatcgcttgtctgtccgtccgtccgtctgtgtggtttctcgaagagggattcagaatcacacacactgctttcctgtagtttagtactgtactttgtgttggtgatattaactacggctgctccggattttccccaatataagggtttcagttatcatgtcaagatgcaagggggtttcacttgctctaatagtcgatcgccgagaagcctgagataatccgatggttgaaagtacagtagtaaattccttgttattcagttggtacaacaatgattatgggtctgacagtaagatgcaacactaagtgggtatagtatgactagcagcgttctctgcttgcgcaaatctagcatgatccggcaatgttctacctgttccctttgtcaggttctc

SEQ ID NO: 11 - DNA, 2026 bp, Triticum aestivum
  source 1..2026: mol_type = unassigned DNA
tgtagtggggatgacacactccggttcaagtccagttgtaggcggggaacttcaacctttgtggcaaacaattcaagtgacttgtcttgcgatccctcaactttatccttgtacacattccaatgcaaatcggaggtgatgggcagagtctttaatcgggacttcactccaacttcaacaccatacctcccaataagcttcacctcatcacttgggttaatccaatgtaacatgcttctcactttcactacaacatcatcatagcttggccttgtgtcaaaaaccatgggctcctcccttccatcggcatttccattcaaaaatgccgccttgtccaagtaatgcaaattaacaagtctatccatctaagaacaatgaaaaaaatttatgtcatccatgggccggccaattcatagagatgaactatccacaatatgcatagattcaagcaagaaacttgtatgcattgcattcaaacaacaggacaaaaattgtcaaaattgccttaaccctaacactaaccctaacccttccaaaacaaagccactaacccttggcacaagatcttgctacccaccacaaagctaggcacaacaaacacaaacataggcataaatgcatcataaatgcacaacaagagcaataattagggtttcaaacaatgtgagcaatctaacataaaatacatgatcccaagcaaccaaaagaagggggagtgggagaggtaccttgggtgatgcaaatgctccggatccaccgaacaaatctctagcaatggagagagatttagtgggtgctttggtgggggagaaagggggaaagaggatgagctcgagaggaagaaggattgagggtgaatgggtgggtgggggccccactagcaacttatccactacagagcctaccaccaaggttgttggcggtaggattgagctccctaccgtcagggtatctggcggtagggcccttaaccacgtaggcgggcgcaaacgaccatctgtgggcagccgtcaaccttaccgctgggatgcttggcggtagcctgtccaacattactgtagtgctgtctgacggtagttgaaagggtcgaatctcaaaatactttcggcgggggtcagatccggttaaatttacggcaaaaagtcaaaacaccaaatttggccgcgggcagccgctgccgtcacgcctgcttctgggaatgatggccgcgtaatttttttttctatgctgtagcctgcagggtggcccatgccatgcgcacgcgtcccctcccagacgcttgaccgagagaccaccatgatttgattctctctccccgactatgatatcagctgcgctccacacagcgaagcccggaggcgaaaagggggccgagcaaggcggcttgtggaccgcccgtcgatcctcttttcactcggcagtccccctcgcatcctccaaaatctcccattccccggttcgttcctaggtgagtctccactttccactcccatgctcgttgtcgcagattcgccggttctgtttcgtttcgcgaatgaagatgtccattttttgggagttgtcaggcgcttgatctgtgggctcgtgggggagatatcctagctgtttagatccgaggcattatgggcatcgatttctgcccatggcttgtctgtccgcctgtctctcgaggatggattcagagtcacagacactgccttcctgttgtttcatttagtattactagtgtcggtgatattaattacggttgctccggatttccccccagcaaaagggtttcaattatcatttcaagatgcaagggggtttcacttgctgtaacaatcgaccgccgagaagcctgagataatgtgatgtctgaaagcacactagtaaattccttgttgttcagttggtacaacaatgattgtgggtctgacaataagatgcaacaataagtgggtatagtacgattagcagcgttttctgcttgcgcaaatctagcatgatccggcaatgttctacctgttccctttgtcaggttctc

SEQ ID NO: 12 - DNA, 2021 bp, Triticum aestivum
  source 1..2021: mol_type = unassigned DNA
tcgggggagctcctattcagcgctataggcgccggttcacgcagctggtgctcgcgcgcgacgtgcagcgggccggcccaacaagctgttgaaaggtaatacgcgaaggaaaaaaaaactccaggtttattcgactcgactgggaatcaaactctggccgccaatttgaatgccacacacgttaaccactaggacaagctcactttgtttaattattagaggaaacaacgttatgtggcccttctttttaataaaatattaaacatatagtagtattatataccagctataaattatttgaataaagaaaacataaatttgaaaaaagcattcacaaaaaccgtgaattgaaagaaatggaaattttgaaaaagatttgtgccaattgaaaaaagtccattgttttgacaaaaaactcatcgatttcgaaaaaaattcatcaaatttgaataaaagttcaccgaattagaaaaaaggttcatgaatttgaatcaagttcatcgactttgaacaaacgttcatcaatttgaataaagttcatcgactttgaaaaaaagttcaccaatttgggaaaaagttaactgattttgaaaaaaattcatcaaatcggaaaaaaatcaaagattttgagaaaaagttcatagattttgaaaaaagttcatcaaactttaaacaaaaatcaacaattttgagaaaaaaatcatcaaatttgaaaaaagttaatcgaatatgaaaaaggttcatcaaatttgaaaaacggtcatcgaaatttaaaaacaattcatcaacttttagaaaaaggttcatcaatttcgcagaaatagctcatcgaaccaggaagaagaaaaaagaaaaaagggaaaaagaaagaaaaagcagaagtaataaaagaatgagaaagatgaacaatatcagacgaggttgtgtggcatggtggttactccactgcgcctcgagctctccccgcacacacttcttttgcgtttttggataccgaagaaaaaaacctacagattaacacagataaatgggccggcccagctaacgcggctgcaggagctggttaggaaaatttcccttaaccggcgcctgcagcgccgaatagggtttgccgctaatccgatcccttcaacccctcgaaagaaaaaatccgactcttcaaaggtgctggcagccgctgacgtcacgcctgcttccggtaatgatggccgcatataattttttttctatgctgtagcctgcagggtggcccatgccatgcgcacgcgtcccctcccagccgctctaccgagagaccaccatgatttgattctctctcccccgactatgatatcagctgtgcgctccacacagcgaagcccggaggcgaaaaggggaccgagcaaggcggctcccggaccgcccgtcgatcctcccctctcattcggcagcccccctcgcatcctccgaaatcccccattccccggttcgttcctaggtgagcttctacttccactcctgcagattcgctggttctgtttcgtttcgcggatgaaaatgtccattttttctggcaaagaaatggttgtcaggcgcttgatctgtggctggtgggggagagatcctagttctttagatccgaggcattatggcatcgattgctgcccatggcttgtctgtccgcctgtctctcgaggatggattcaaactcaggcactgctttcctgtagtttagtatactagtgtcggtgatgttaattacggttgctccggatttccccccagcaaaagggtttcaattatcatttcaagatgcaagggggtttcacttgctgtaacagtcgaccgccgagaaccctgagataatgtgatgtctgaaagtacactagtaaattccttgttattcagttggtagaacaatgattgtgggtctgacaataagatgcaacactaagtggctatagtatgattagcagcgttttctgcttgcgcaaatctagcatgatccggtaatgttctacctgttcccttgtcaggttctc

SEQ ID NO: 13 - DNA, 47 bp, Triticum aestivum
  source 1..47: mol_type = unassigned DNA
gaaggtgaccaagttcatgcttgccaaatggttgaagatcaataagg

SEQ ID NO: 14 - DNA, 21 bp, Triticum aestivum
  source 1..21: mol_type = unassigned DNA
gaaggtgaccaagttcatgct

SEQ ID NO: 15 - DNA, 49 bp, Triticum aestivum
  source 1..49: mol_type = unassigned DNA
gaaggtcggagtcaacggattgttgccaaatggttgaagatcaataaga

SEQ ID NO: 16 - DNA, 21 bp, Triticum aestivum
  source 1..21: mol_type = unassigned DNA
gaaggtcggagtcaacggatt

SEQ ID NO: 17 - DNA, 30 bp, Triticum aestivum
  source 1..30: mol_type = unassigned DNA
caatactcatcaatgacaaaagctgcgaat

SEQ ID NO: 18 - DNA, 44 bp, Triticum aestivum
  source 1..44: mol_type = unassigned DNA
gaaggtgaccaagttcatgcttttcctctatcttgccactgcag

SEQ ID NO: 19 - DNA, 21 bp, Triticum aestivum
  source 1..21: mol_type = unassigned DNA
gaaggtgaccaagttcatgct

SEQ ID NO: 20 - DNA, 46 bp, Triticum aestivum
  source 1..46: mol_type = unassigned DNA
gaaggtcggagtcaacggattgttttcctctatcttgccactgcaa

SEQ ID NO: 21 - DNA, 21 bp, Triticum aestivum
  source 1..21: mol_type = unassigned DNA
gaaggtcggagtcaacggatt

SEQ ID NO: 22 - DNA, 30 bp, Triticum aestivum
  source 1..30: mol_type = unassigned DNA
agtcatattatcaggatcaacttcatcctt

SEQ ID NO: 23 - DNA, 20 bp, Triticum aestivum
  source 1..20: mol_type = unassigned DNA
attgcttgtcgccagatcca

SEQ ID NO: 24 - DNA, 20 bp, Triticum aestivum
  source 1..20: mol_type = unassigned DNA
aagggttctgagctaacgcc

SEQ ID NO: 25 - DNA, 20 bp, Triticum aestivum
  source 1..20: mol_type = unassigned DNA
atgcgcacatgtgtttgcat

SEQ ID NO: 26 - DNA, 20 bp, Triticum aestivum
  source 1..20: mol_type = unassigned DNA
ctcaggcaacccagaggaag